(19) United States
(12) Patent Application Publication
(10) Pub. No.: US 2011/0034654 A1
(43) Pub. Date: Feb. 10, 2011

(54) POLYMER ORGANOCATALYST AND PREPARATION PROCESS

(76) Inventors: Tore Hansen, Oslo (NO); Finn Knut Hansen, Strommen (NO); Tor Erik Kristensen, Oslo (NO)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/812,315

(22) PCT Filed: Jan. 7, 2009
(86) PCT No.: PCT/EP09/50141

§ 371 Date: Oct. 4, 2010

(30) Foreign Application Priority Data
Jan. 9, 2008 (GB) 0800324.6

(51) Int. Cl. C08F 126/06; C07C 205/45

(52) U.S. Cl. 526/258; 568/306

(57) ABSTRACT

A chiral polymer organocatalyst comprising a main chain and side chain organocatalytic groups covalently attached to the main chain, which organocatalytic groups comprise an amino acid or amino acid derivative of the following general formula (I), in which one stereoisomeric form predominates: formula (I) wherein the catalyst is bound to the polymer main chain via R¹, R², R⁴, R⁵ or R⁶ through a linker (L) or direct bond, and wherein R¹-R⁶ and Z are defined as follows: R¹ is H, a naturally occurring alpha-amino acid side chain or a non-natural commercially available alpha-amino acid side chain that may contain L; R² is H, O (doubly bonded to give a carbonyl), O-L (where L is a linker), NH-L or L; R³ is H or doubly bonded to give a carbonyl with R² when R² is O; R⁴ is H, C₁-C₆ alkyl or L R⁵ is H, CO₂H, C₁-C₆ alkyl, benzyl, L, CONHR (in which R is alkyl, aryl, heteroaryl, arylalkyl or, heteroarylalkyl), tetrazolyl, CH2 coupled to a triazole moiety, an esterified CH₂OH or CO₂R (in which R is alkyl, aryl, heteroaryl, arylalkyl N or heteroarylalkyl), formula (II) or formula (III) wherein z is formula (IV) or a directed bond, X₄ is H, Me₃Si or Et₃Si, X₃ comprises a naturally-occurring alpha-amino acid side chain, H, C₁-C₆ alkyl or phenyl, Ar₁ and Ar₂ are each independently aryl or heteroaryl, and Y denotes the position of attachment to the main chain or linker; and R⁶ is H, CO₂H3 C₁-C₆ alkyl, benzyl or L; and wherein the polymer organocatalyst comprises a cross-linked polymer.

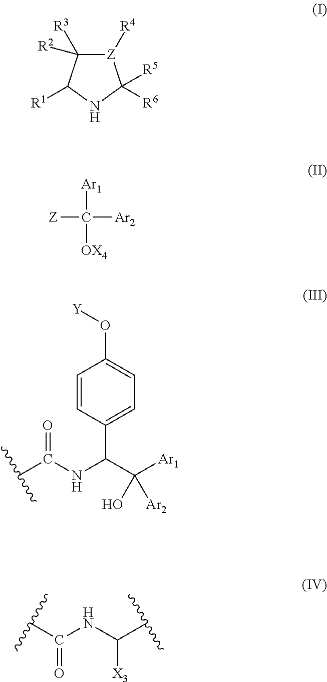

### TECHNICAL FIELD

The present invention relates to a chiral polymer organocatalyst, a process for the preparation thereof, and use of the polymer organocatalyst in asymmetric organic transformations.

### BACKGROUND OF THE INVENTION AND DESCRIPTION OF RELATED ART

In the field of synthetic organic chemistry, organocatalytic reaction systems, especially asymmetric organocatalytic reaction systems, have gained considerable importance during the last decade. In these systems, relatively low-molecular weight organic compounds (and also relatively large compounds in some cases) are used as catalysts in asymmetric chemical transformations. These systems, as opposed to the more classical transition metal-based catalytic systems, have the advantage of being more environmentally friendly and less toxic, as well as often being tolerant to a very wide variety of different reaction conditions, such as the presence of water and air. They do not pollute products with traces of heavy metals, and these metal-free catalysts are also often of a very convenient, robust and simplistic nature, making their large scale preparations economical. Their benign toxicity offers advantages over existing systems for asymmetric synthesis.

Of the organocatalytic reaction systems, the organocatalysts based on the readily available amino acid L-proline (1), and derivatives synthesized using proline, have together with the organocatalysts belonging to the class of the imidazolidin-4-ones (2), shown the greatest versatility and widest applications. These systems have shown the ability of catalyzing a very wide range of synthetically useful organic transformations, such as aldol reactions (intramolecular, intermolecular and modified), Friedel-Craft alkylations, α-fluorinations, α-chlorinations, α-aminations, α-aminooxylations, Diels-Alder reactions, 1,3-dipolar cycloadditions, Mannich reactions and Michael additions (encompassing subtypes in many of these categories), often with a very high degree of asymmetric induction. This makes them potentially very useful for the production of valuable chemical products or intermediates, such as, but not limited to, those within the pharmaceutical, agrochemical and other fine chemical industries.

Known organocatalytic reaction systems suffer from some notable disadvantages. The systems very often require substantial catalyst loadings, of the order of 10-35 mol % or even larger, making catalyst preparation and recycling of significant importance. In addition, the desired products are sometimes very difficult to separate from the catalyst used in these reactions because their chemical properties often resemble those of the other constituents of the reaction system. In attempts to address these difficulties, organic chemists have immobilized organocatalysts on solid supports such as polymer or silica particles, creating a heterogeneous system which can be conveniently filtered off or centrifuged after completed reaction and then purified and reused. The organocatalyst can also be attached to a completely soluble macromolecule, which can be precipitated after reaction by addition of a suitable solvent and then filtered off and reused in the same way as the solid supports.

An organocatalyst can be fitted with an appropriate functional group, capable of binding the catalyst onto a prefabricated solid support or macromolecule. This approach has been described by Benaglia et al. (Adv. Synth. Catal. 2001, 343, 171-173 and Adv. Synth. Catal. 2002, 344, 533-542), who used polyethylene glycol (PEG) of average molecular weight 5000 to immobilize proline by esterification of the 4-hydroxy group of trans-4-hydroxy-L-proline (3) with PEG. The immobilized catalyst was used in a homogeneous reaction system to induce asymmetry in the preparation of β-hydroxy ketones and β-amino ketones. The catalyst was recovered by addition of diethyl ether and subsequent filtration of the precipitated catalyst. In a very analogous way, proline has been immobilized onto the microporous Merrifield resin (Gruttadauria et al., Eur. J. Org. Chem. 2007, 4688-4698 and Font et al., Org. Lett. 2006, 8, 4653-4655), modified polystyrene resins (Andreae et. al., Tetrahedron Asym. 2005, 16, 2487-2492), linear polystyrene (Liu et al., Tetrahedron Asym. 2007, 18, 2649-2656) or mesoporous silica (Doyagüez et. al., J. Org. Chem. 2007, 72, 9353-9356) by analogous methods. This work has recently been reviewed by Gruttadauria et al. (Chem. Soc. Rev. 2008, 37, 1666-1688). These immobilized catalysts are reputed to be successful at inducing asymmetry in many of the same reactions as the organocatalysts are capable to do in its free, non-immobilized form, such as aldol and imino-aldol reactions.

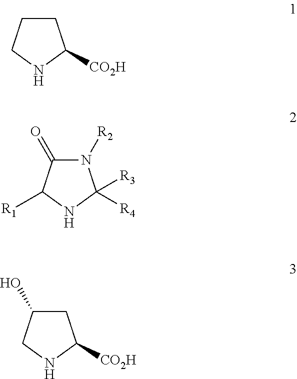

In much the same way as have been disclosed for proline, organocatalysts belonging to the class of the imidazolidin-4-ones (2) have been immobilized onto modified polystyrene resin (Selkälä et al., Adv. Synth. Catal. 2002, 344, 941-945), PEG (Puglisi et al., Eur. J. Org. Chem. 2004, 567-573) or siliceous mesocellular foams (Ying et. al., WO 2007/084075 A1) and used for asymmetric induction in Friedel-Craft alkylations, Diels-Alder cycloadditions and 1,3-dipolar cycloadditions.

All of the above mentioned systems have been devised by organic chemists to be effective in promoting asymmetric induction. However they suffer from one or more major disadvantages. Their preparation is inherently complicated, often using lengthy procedures on small scale. The tolerance of the immobilized catalysts to variations in reaction conditions is very limited since catalyst preparation is restricted to a rather small number of commercially available solid supports. Accordingly, these catalysts are for the most part only useful for academic pursuits. In addition to this, there are also very limited possibilities of achieving high catalyst loadings on rigid macroporous polymer supports by the conventional immobilization procedures.

In the field of polymer chemistry US2004/0082463 is directed to phase selective polymer supports for catalysis. These catalysts use polystyrene copolymers having enhanced solubility in non-polar solvents to provide catalytic methods that allow for the efficient separation of the catalyst from the reaction product and recycling of the catalyst with minimal additional solvent to effect separation. Styrene monomers substituted with catalytic species are described in which the catalytic species can be an organic group or a metal complex. No functional monomers of any interest for asymmetric organocatalysis are disclosed. The styrene monomers of this disclosure usually have to be prepared with vinylbenzyl chloride, which is a hazardous material only available at high cost when high purity is required. Alternatively, derivatisation of polystyrene by chloromethylation generally requires highly carcinogenic chloromethyl methyl ether. Moreover, this disclosure teaches the use of benzoyl peroxide as an initiator in preparation of the polymers, which would have been expected to react undesirably with many amine-containing organocatalysts.

US 2004/0198591 is directed to a proposal for a polymer-bound catalyst for the enantioselective aldol or mannich reaction. Polymer-enlarged chiral catalysts which comprise prolines or proline analogues are proposed so that the catalyst dissolves in the solvent to be used. Polymer enlargement may be achieved by copolymerisation of a monomer which comprises an active catalytic unit or by binding the active unit to a finished polymer. However, no worked examples are provided in this proposal and the effectiveness of such polymer-enlarged catalysts is questioned by the present applicants who have found soluble polymer organocatalysts to be largely ineffective in asymmetric organic transformations.

### SUMMARY OF THE INVENTION

The present invention aims to solve the problems of the prior art by providing in a first aspect a chiral polymer organocatalyst comprising a main chain and side chain organocatalytic groups covalently attached to the main chain, which organocatalytic groups comprise an amino acid or amino acid derivative of the following general formula, in which one stereoisomeric form predominates:

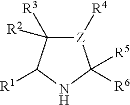

wherein the catalyst is bound to the polymer main chain via R¹, R², R⁴, R⁵ or R⁶ through a linker (L) or direct bond, and wherein R¹-R⁶ and Z are defined as follows:
R¹ is H, a naturally occurring alpha-amino acid side chain or a non-natural commercially available alpha-amino acid side chain that may contain L;
R² is H, O (doubly bonded to give a carbonyl), O-L (where L is a linker), NH-L or L;
R³ is H or doubly bonded to give a carbonyl with R² when R² is O;
R⁴ is H, C₁-C₆ alkyl or L
R⁵ is H, CO₂H, C₁-C₆ alkyl, benzyl, L, CONHR (in which R is alkyl, aryl, heteroaryl, arylalkyl or, heteroarylalkyl), tetrazolyl, CH₂ coupled to a triazole moiety, an esterified CH₂OH or CO₂R (in which R is alkyl, aryl, heteroaryl, arylalkyl or heteroarylalkyl)

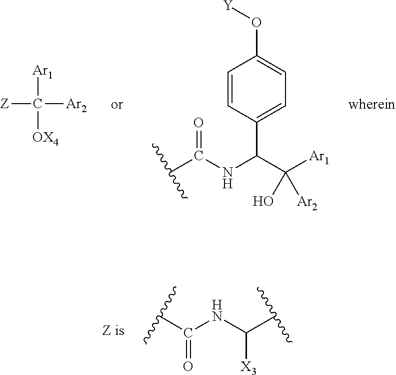

or a direct bond,

X₄ is H, Me₃Si or Et₃Si, X₃ comprises a naturally-occurring alpha-amino acid side chain, H, C₁-C₅ alkyl or phenyl, Ar₁ and Ar₂ are each independently aryl or heteroaryl, and Y denotes the position of attachment to the main chain or linker; and

R⁶ is H, CO₂H, C₁-C₅ alkyl, benzyl or L; and
wherein the polymer organocatalyst comprises a cross-linked polymer.

In a further aspect, the present invention provides a process for the preparation of a chiral polymer organocatalyst, which process comprises:
providing monomers comprising an organocatalytic group covalently attached to a polymerisable unit; and polymerising the polymerisable units to form the polymer organocatalyst; wherein the organocatalytic group comprises

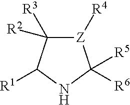

wherein the catalyst is bound to the polymer main chain via R¹, R², R⁴, R⁵ or R⁶ through a linker (L) or direct bond, and wherein R¹-R⁶ and Z are defined as follows:
R¹ is H, a naturally occurring alpha-amino acid side chain or a non-natural commercially available alpha-amino acid side chain that may contain L;
R² is H, O (doubly bonded to give a carbonyl), O-L (where L is a linker), NH-L or L;
R³ is H or doubly bonded to give a carbonyl with R² when R² is O;
R⁴ is H, C₁-C₆ alkyl or L
R⁵ is H, CO₂H, C₁-C₆ alkyl, benzyl, L, CONHR (in which R is alkyl, aryl, heteroaryl, arylalkyl or, heteroarylalkyl), tetrazolyl, CH₂ coupled to a triazole moiety, an esterified CH₂OH or CO₂R (in which R is alkyl, aryl, heteroaryl, arylalkyl or heteroarylalkyl)

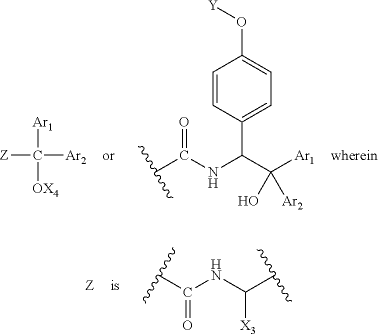

or a direct bond,
X₄ is H, Me₃Si or Et₃Si, X₃ comprises a naturally-occurring alpha-amino acid side chain, H, C₁-C₅ alkyl or phenyl, Ar₁ and Ar₂ are each independently aryl or heteroaryl, and Y denotes the position of attachment to the main chain or linker; and
R⁶ is H, CO₂H, C₁-C₅ alkyl, benzyl or L; and
wherein the step of polymerizing includes cross-linking polymer main chains.

The present invention overcomes a number of disadvantages of the prior art. By constructing the asymmetric organocatalysts in such a way that a structural unit capable of polymerisation is included, polymers produced from the monomers comprising the organocatalytic group are themselves used to create polymeric supports. In this way, a very wide selection of polymeric supports may be used as building blocks for an asymmetric organocatalytic reaction system. Great flexibility in polymer characteristics and morphology is available and catalyst synthesis and operation may be performed in an environmentally friendly way. By incorporating the organocatalytic groups as side chains on a main chain polymer, a much higher catalyst loading is achievable as compared with the prior art. Typically, a loading of active catalyst of up to about 5.4 mmol/g total catalyst may be achieved. A loading as low as 0.05 mmol/g or even lower may be used, although it is in most cases preferred to use more than 0.2 mmol/g. A loading above 0.6 mmol/g, preferably at least 1 mmol/g is achievable in most cases. This is in contrast to most of the prior art loadings of 0.5-0.6 mmol/g.

The present applicants have found that soluble polymer systems incorporating the organocatalytic groups described herein work poorly as organocatalysts. However, cross-linked chiral polymer organocatalysts according to the invention have been found to work surprisingly well in asymmetric organic transformations. The cross-linked polymer organocatalysts are never completely homogenously soluble in all solvents. Having a cross-linked network allows the formation of a swelled and gel-like bead or particle in solution that can be filtered and re-used, thereby avoiding the need for membrane filtration or precipitation which are used for soluble polymers. In addition, immobilized catalysts regularly outperform the monomeric catalysts with regards to selectivity. The polymer scaffold is therefore not only a convenient tool for immobilization, but plays an active part in the outcome of the reactions, often enhancing stereoselectivity.

Many of the problems related to classical immobilization procedures are related to the fact that the chemistry used for the catalyst preparations is for the majority of them taking place on a non-soluble support where analysis of what chemistry has actually taken place is very limited when compared to soluble non-polymeric compounds. In addition, such polymer supports show an extensive swelling in solvent systems, this being necessary for their function, but inconvenient in their preparation, most often over several steps, since it may necessitate a disproportionally large amount of potentially costly and/or toxic solvent during their manufacture. According to the present invention, a more general approach is provided for immobilization of organocatalysts, an approach more applicable for large scale manufacture. In order to provide a wider variability of solid supports/soluble macromolecules and having synthetic procedures more applicable to large scale manufacture, a novel way of constructing asymmetric organocatalysts is provided.

According to the present invention, the chiral polymer organocatalyst may be used in asymmetric organic transformations. It is for this reason that the amino acid or amino acid derivative of the organocatalytic groups is present. Various exemplary asymmetric organic transformations are discussed in further detail below. Generally, the amino group of the amino acid or amino acid derivative participates in these transformations by forming enamine or iminium intermediates, which by virtue of their nature are capable of reacting with a wide range of substrates to give products that are capable of releasing their amine functionality to provide a catalytic cycle. The amino acid or amino acid derivative is chiral. In order to achieve asymmetric organic transformation, one stereoisomeric form of the amino acid or amino acid derivative must predominate so that one stereochemistry of transformation is favoured over another. Generally, there is at least 60% of one stereoisomeric form, advantageously at least 70%, preferably at least 80%, more preferably at least 90% and most preferably at least 95% of one stereoisomeric form. In a particularly preferred embodiment there is approximately 100% of one stereoisomeric form. Depending on the stereochemistry desired in a particular transformation, this form may be R or S.

The organocatalytic groups are covalently attached to the polymer main chain through any of the substituent groups R¹, R², R⁴, R⁵ or R⁶. This attachment may be direct or via a linker as discussed in further detail below. The point of attachment on a substituent group may be determined empirically and examples are described herein. Primarily, the attachment should not interfere with the catalytic activity of the organo catalytic group.

The organocatalytic groups of the invention are preparable from amino acids and contain an amine group which is important in catalysis because an enamine or iminium species may be formed with the substrate. The organocatalytic group contains a 5-membered ring which is a pyrrolidine ring when Z is CH or an imidazolidinone ring when Z is N.

In one arrangement, Z is CH and R² is attached to the main chain, optionally via a linker. In this arrangement it is preferred that R⁵ is CO₂H and R¹, R⁴ and R⁶ are each H. This arrangement is described in further detail below as a Type 1 polymer. In other arrangements, R¹ may be a naturally occurring alpha-amino acid side chain. By naturally-occurring alpha-amino acid side chain is meant the side chain groups of any of the amino acids found in nature (although the stereochemistry might not be the same as found in nature). These amino acids include serine, threonine, cysteine, tyrosine, asparagine, glutamine, aspartic acid, glutamic acid, lysine, arginine, histidine, alanine, valine, leucine, isoleucine, phenylalanine, tryptophan and methionine.

There are further arrangements in which it is preferred that Z is CH and R¹, R⁴ and R⁶ are each H. According to one arrangement, R⁵ is

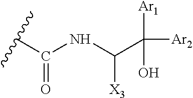

Here it is preferred that R³ is also H. In this way a Type 2 polymer is formed, as described below. Alternatively, R⁵ is

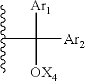

It is preferred that R³ is H, whereby a Type 3 polymer is formed, as described below.

In a further arrangement where Z is CH, R⁵ comprises

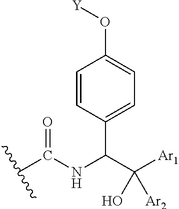

Here it is preferred that R¹ and R³ are also H.

According to this embodiment, the point of attachment to the polymer main chain is not from the pyrrolidine ring but instead from position Y. This is exemplified as a Type 4 polymer discussed below.

In a further embodiment, an imidazolidinone ring is formed in which Z is N and R² and R³ together form carbonyl. In this arrangement, it is preferred that R¹ is attached to the polymer main chain, optionally by a linker. R⁴ may be C₁ to C₆ alkyl, and R⁵ and R⁶ may each independently be C₁ to C₆ alkyl, benzyl or carboxylate. These polymers are discussed in further detail below as a Type 5 polymer.

In an alternative arrangement, Z is N, and R² and R³ together form a carbonyl wherein R⁴ is attached to the main chain, optionally by a linker. R⁵ and R⁶ may each independently be C₁ to C₆ alkyl, benzyl or carboxylate and R¹ is Ar₁—CH₂. These polymers are discussed in further detail below as Type 6 polymers.

In a preferred arrangement, each amino acid or amino acid derivative is attached to the main chain via a linker which typically comprises a linear or branched hydrocarbylene, aliphatic or aromatic, which may optionally be substituted with one or more heteroatoms and different functional groups, and may incorporate one or more rings. The chain length of the linker is preferably in the range of from 2 to 25 atoms, more preferably 2-10 atoms. The purpose of the linker is to ensure that the organocatalytic groups are spaced sufficiently apart from the polymer main chain so that they are accessible to reactants. Any sort of molecular moiety may be used as a linker to provide increased molecular distance between the asymmetric organocatalytic groups and the polymer main chain. Synthesis of such a structural moiety can be exemplified by the use of difunctional structural units, of the aliphatic or aromatic type, such as γ-hydroxybutyric acid, malonic acid, succinic acid, adipic acid, para-aminobenzoic acid or any other unit that can be thought of as having one functional moiety for binding to the organocatalytic group and one functional moiety for binding to the main chain. An ethyl succinoyl linker is particularly useful. This may be provided as 2-methacryloyloxyethyl succinic acid or a derivative thereof. A linker can also be thought of as a two-part structural fragment, consisting of two molecular units where one of them is fitted onto the polymerisable unit and the other to the structural unit containing the organocatalytic group. Then the two fragments are reacted with each other through one or more chemical reactions, providing a linkage between the two of them and in such a way providing a linker. An example of such a linker is one where one of the units are provided with an azide-group and the other with an alkyne-moiety and the two are joined together in a Huisgen-type copper catalyzed cycloaddition reaction, this being an example of the well-known “click-chemistry”. The linker also takes part in providing hydrophilic/lipophilic characteristics to the polymer system in question and in this way affects the chemical characteristics of the polymeric catalyst.

It is preferred that the main chain polymer comprises a polyacrylate or polymethacrylate. Although other polymers such as polystyrenes may be used as the main chain polymer, (meth)acrylate polymers are preferred because a vast array of morphologies may be produced and because the production process for forming the polymers will have a broader basis of available starting materials.

Monomers may be polymerised in a radical polymerization with or without co-monomers to obtain the polymer systems of the invention. These polymer systems can be either cross-linked homo- or co-polymers prepared by bulk, solution, dispersion/precipitation, suspension (normal or inverse) or emulsion (normal or inverse) polymerization and used as such, or systems using polymer particles created in such systems as seed particles in a suspension polymerization, or any other type of cross-linked microporous or macroporous particles or other cross-linked structures (such as monoliths) with chemical characteristics and catalyst loadings that are suited for organocatalytic reactions. As used herein, the term macroporous polymers refer to polymer systems having macropores. In the present context, the macropore means pores with average diameter about 3.5 to 10 000 nm. Micropore refers to pores of average diameter from about 0.10 to about 3.5 nm. The polymer particles can be of the polydisperse or monodisperse types, created by suspension (normal or inverse), microsuspension, emulsion (normal or inverse), miniemulsion, dispersion or seeded polymerization or any other type of radical polymerization that is suited for the preparation of the support in question.

The polymerization of (meth)acrylic monomers may also be used to derivatise polymer beads of either the microporous or macroporous type, including monodisperse ones, by grafting the polymer chains onto prefabricated polymer beads of the desired type and characteristics.

In one embodiment, the main chain polymer comprises a copolymer.

The co-monomers used together with the monomers of this invention in a co-polymerization can be synthesized from or be taken directly from the large assortment of commercially available monomer units that are able to undergo a radical polymerization. Such an assortment is certainly not limited to, but exemplified by acrylic acid and its derivatives such as acryloyl halides (e.g. acryloyl chloride), alkyl acrylates (e.g. methyl, ethyl and butyl acrylate), acrylonitrile and acrylamides, by methacrylic acid and its derivatives such as methacryloyl halides (e.g. methacryloyl chloride), alkyl methacrylates (e.g. methyl, ethyl and butyl methacrylate), 2-hydroxyethyl methacrylate (HEMA) and glycidyl methacrylate (GEMA), by non-halogenated or halogenated dienes such as butadiene, isoprene and chloroprene, by monoethylenically unsaturated monomers such as vinyl acetate, maleic acid, maleic anhydride, dimethyl maleate, diethyl maleate, dibutyl maleate, fumaric acid, dimethyl fumarate, diethyl fumarate and vinyl chloride, and by vinylaromatic compounds such as vinylpyridine, vinylphenol, vinylnaphthalene, vinylanthracene, styrene, alkylstyrenes (e.g. methyl, ethyl, dimethyl and ethyl methyl styrene), halostyrenes (e.g. p-chlorostyrene, 2,4-dichlorostyrene, m-fluorostyrene), 3-nitrostyrene, vinylbenzyl chloride, aminostyrenes and other derivatives alike. Such an assortment also includes various bi- or higher order functional monomers, in this way providing a crosslinked structure, such as exemplified by ethyleneglycol dimethacrylate (EGDMA), butanediol diacrylates, ethylene glycol diacrylate, polyethylene glycol diacrylates, polyethylene glycol dimethacrylates, polypropylene glycol diacrylates, tetraethylene glycol diacrylate, N,N′-methylenebisacrylamide, pentaerythritol trimethacrylate, the polyvinylethers of glycol, glycerol, penta-erythritol and resorcinol and the polyvinylaromatic hydrocarbons such as divinylbenzenes, divinyltoluenes, divinylxylenes, divinylnaphthalenes and divinylethylbenzene. It is also to be understood that the monomers may also consist of more specialized monomers to give the finished polymer system the desired characteristics, such as halogenated ones (e.g. pentabromobenzyl acrylate, 2- and 3-trifluoromethylstyrene, pentafluorostyrene, 2,2,2-trifluoroethyl acrylate and various other polyfluorinated alkyl acrylates) and the like. The provided assortment of examples is not in any way thought of as being exhaustive and limiting to the scope of the invention, but rather to exemplify the innovations provided by the current invention.

### DETAILED DESCRIPTION OF THE INVENTION

The present invention will now be described in further detail, by way of example only, with reference to the following Examples.

A first aspect of the invention is to produce (meth)acrylic monomers, containing the desired asymmetric organocatalyst, which are suited for radical polymerization with or without co-monomers. According to the present invention, the (meth)acrylic monomer of this invention belongs to one of the general types 1-6, depicted below.

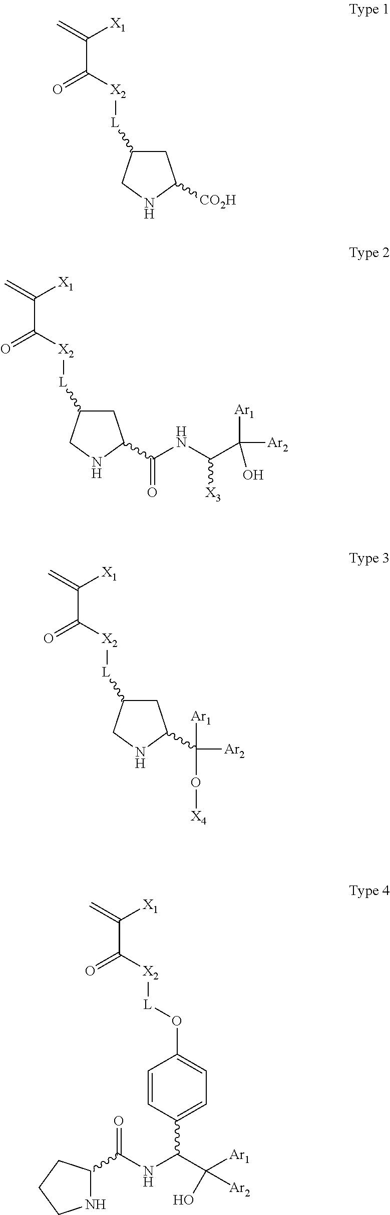

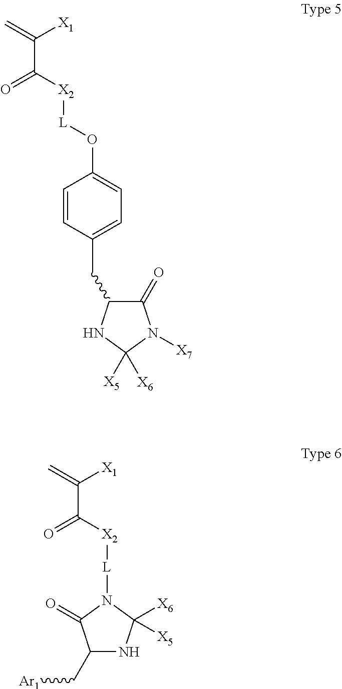

The general descriptors are defined as follows:

Wavy lines indicate bonds where the absolute configuration of chiral centers is not specified and can be of both types possible at that site and encompasses any combination of such chiral centers when the monomer contains several of them.

X₁=Hydrogen or methyl (specifying acrylate/methacrylate).

X₂=O or NH (specifying acrylic or methacrylic esters or amides respectively).

X₃=α-Amino acid side chain, both natural and non-natural, such as hydrogen, methyl, ethyl, n-propyl, iso-propyl, n-butyl, iso-butyl, sec-butyl, tert-butyl, benzyl, phenyl and the like.

X₄=Hydrogen, trimethylsilyl or triethylsilyl.

X₅=Alkyl such as methyl, ethyl, n-propyl, iso-propyl, n-butyl, sec-butyl, tent-butyl, iso-butyl, pentyl, neopentyl, benzyl and the like or carboxylic acid (—CO₂H). In the simplest form, X₅=X₆=methyl. In addition, X₅ and X₆ can together be part of the same common cycloalkyl, forming rings of 5, 6 or any other suitable ring size. In addition, X₅ may be a heteroaromatic group such as furyl.

X₆=Alkyl such as methyl, ethyl, n-propyl, iso-propyl, n-butyl, sec-butyl, tert-butyl, iso-butyl, pentyl, neopentyl, benzyl and the like or carboxylic acid (—CO₂H). In the simplest form, X₅=X₆=methyl. In addition, X₅ and X₆ can together be part of the same cycloalkyl, forming rings of 5, 6 or any other suitable ring size. In addition, X₆ may be a heteroaromatic group such as furyl.

X₇=Alkyl such as methyl, ethyl, n-propyl, iso-propyl, n-butyl, sec-butyl, tert-butyl, iso-butyl, pentyl, neopentyl and the like.

Ar₁=Aryl or heteroaryl such as phenyl, mono- and polyhalophenyl, alkylphenyl, alkoxyphenyl, trifluoromethylphenyl, 3,5-bis(trifluoromethyl)phenyl, naphtyl, anthracyl, pyridyl, furyl, indolyl and the like. In the simplest form, Ar₁=Ar₂=phenyl.

Ar₂=Aryl or heteroaryl such as phenyl, mono- and polyhalophenyl, alkylphenyl, alkoxyphenyl, trifluoromethylphenyl, 3,5-bis(trifluoromethyl)phenyl, naphtyl, anthracyl, pyridyl, furyl, indolyl and the like. In the simplest form, Ar₁=Ar₂=phenyl.

L=Linker (optional).

Monomers of the general type 1 are prepared starting from the commercially available amino acid 4-hydroxyproline. trans-4-Hydroxy-L-proline (3) is a major component of the protein collagen, playing key roles for collagen stability. It is a relatively major product of commerce, and as such, is a natural starting point for any utilization of proline for binding as part of a larger catalyst system. Any other stereoisomer of 4-hydroxyproline, of which most are commercially available, can be utilized in the same manner. The hydroxyl group can be conveniently and efficiently transformed and linked as part of a larger molecular arrangement, leaving the amino acid functionalities of proline untouched and available for catalytic activity. The simplest monomer, the O-acryloyl-trans-4-hydroxy-L-proline, can be prepared in two ways. trans-4-Hydroxy-L-proline can be fitted with a protecting group for the amino functionality and then acylated with the acid chloride or acid anhydride in a suitable organic solvent and finally deprotected to give the desired acrylic monomer. The other derivatives can be prepared by analogy. The protecting group can be any of the many available for the protection of the amino group, such as but certainly not limiting to, tert-butoxycarbonyl-(Boc), carbobenzoxy-(Cbz), 9-fluorenyl-methoxycarbonyl-(Fmoc) or allyloxycarbonyl-(alloc) protecting group, or other ones known for one skilled in the art, and can be readily found in chemical literature such as Greene's Protective Groups in Organic Synthesis by Wuts and Greene (4^{th} Ed., Wiley, 2006), incorporated herein by reference. For some uses, it may be necessary to protect the carboxylic acid function as well. The purpose of such a protection is not only to mask one or both of these functionalities during subsequent synthetic transformations, but also to allow the otherwise overwhelmingly hydrophilic hydroxyproline to become more lipophilic and in such a way making it available for synthetic procedures taking place in the environment of an organic solvent system. In addition, the subsequent radical polymerization, which may take place in any of the several systems available for such polymerization, may require specific physical characteristics, most notably solubility, that is compatible with the polymerization system utilized.

In the preparation of O-acryloyl-trans-4-hydroxy-L-proline, we have found that conventional procedures for preparation of such an amino acid side chain (meth)acrylic monomer, as disclosed in WO 2006/126095 A2, using copper complex protection, does not work for hydroxyproline because of the unreactive nature of the secondary alcohol, nor is any experimental details, spectroscopic data or use of the claimed monomer/polymer provided. We have found that O-acryloyl-trans-4-hydroxy-L-proline can be efficiently prepared by direct acylation of hydroxyproline with acryloyl chloride in a highly acidic medium consisting of neat trifluoroacetic acid containing a catalytic amount of trifluoromethanesulfonic acid without the need for any protective group chemistry or chromatography. Crystallization of product is initiated by addition of diethyl ether. Optionally, the acylation can take place in neat methanesulfonic acid, although the product is then separated after addition of diethyl ether in a less practical oily form. This protocol offers an attractive alternative to the protective group approach outlined above.

In the preparation of monomers of the general type 2, procedures may be built upon the work of Raj et al. (Org. Lett. 2006, 8, 4097-4099) and Wu et al. (Tetrahedron Asym. 2000, 11, 3543-3552). Optically active β-amino alcohols may be prepared by treating alkyl esters of the appropriate amino acid, or their corresponding salts, with an excess of a suitable Grignard-reagent. The resultant optically active β-amino alcohols may then be coupled via an amide linkage to a protected proline derivative, using any of the many methods available for such a reaction, such as, but not limited to, the use of mixed anhydrides prepared from alkyl chloroformates. The proline amide may then be deprotected. Such a procedure can be exemplified by the treatment of an amino acid such as L-phenylalanine with a slight excess of anhydrous hydrogen chloride, prepared in situ with the aid of thionyl chloride at 0-4° C., in methanol overnight and evaporation of volatiles under reduced pressure to give L-phenylalanine methyl ester hydrochloride. This may then be treated with 8-10 equivalents of phenylmagnesium bromide in THF at 0° C. to room temperature for 5-24 h and subsequently recrystallized from ethanol to give pure (S)-2-amino-1,1,3-triphenylpropan-1-ol. A solution of N-(benzyloxycarbonyl)-L-proline in dichloromethane at 0° C. may then be treated with one equivalent of triethylamine, followed by one equivalent of ethyl chloroformate. After stirring for 15 min, slightly less than one equivalent of the optically pure β-amino alcohol may be added and the solution stirred for 5 h. Work-up and recrystallization from ethyl acetate gives the N-(benzyloxycarbonyl)-L-prolinamide, which can be deprotected in excess neat formic acid for 10 h at 0° C., followed by neutralization with solid sodium hydrogen carbonate and extraction with ethyl acetate to give a catalyst of the general type 2 without the (meth)acryloyl moiety. In the preparation of monomers of the general type 2, proline can be substituted with hydroxyproline, where the (meth)acryloyl functionality has already been introduced onto position 4 by methods already discussed and the procedure adapted to give a monomer suitable for polymerization.

In the preparation of monomers of the general type 3, procedures may be built upon the work of Jørgensen et al. (Angew. Chem. Int. Ed 2005, 44, 794-797 and US2007/0276142 A1) and Kanth et. al. (Tetrahedron 1993, 49, 5127-5132). An alkyl ester of proline, its corresponding salts or a carbamate of an alkylester, of proline may be treated with an excess of the appropriate Grignard-reagent and appropriately hydrolyzed to give an optically active prolinol. Then, if desired, the alcohol can be silylated with a silylating reagent such as, but not limiting to, trimethylsilyl trifluoromethanesulfonate. Such a procedure can be exemplified by treating proline with potassium carbonate and just over two equivalents of ethyl chloroformate in methanol at room temperature for 18 h. Evaporation of solvent, addition of water and extraction with dichloromethane gives the ethyl carbamate of proline methyl ester. A Grignard-reagent may be prepared from equimolar amounts of magnesium and 2,5-bis(trifluoromethyl)bromobenzene in THF at reflux for 1 h. Just over two equivalents of this reagent may be reacted with a solution of the ethyl carbamate of the proline methyl ester at 0° C., allowed to reach room temperature and refluxed for 2 h. Normal work-up with ammonium chloride and recrystallization from diethyl ether give a crystalline product that is hydrolyzed by ten equivalents of potassium hydroxide in methanol at reflux for 2 h. Work-up, silylation of the product with 1½ equivalents of trimethylsilyl trifluoromethanesulfonate and triethylamine in dichloromethane until full conversion, as judged by thin layer chromatography, and purification by column chromatography on silica gel with pentane/dichloromethane gives (S)-2-[bis-(3,5-bistrifluoromethylphenyl)-trimethylsilanyloxymethyl]-pyrrolidine. In the preparation of monomers of the general type 3, proline can be substituted with hydroxyproline, where the (meth)acryloyl functionality is introduced onto position 4, by methods already discussed, after the treatment with Grignard-reagent, and in such a way to give a monomer suitable for polymerization.

In the preparation of monomers of the general type 4, procedures may be built upon the work already discussed for monomers of the general type 2, except that tyrosine may be used as the starting amino acid and proline can be used instead of hydroxyproline. In addition, the (meth)acryloyl-moiety can be introduced either before or after the amide linkage is prepared by the appropriate procedures.

In the preparation of monomers of the general types 5 and 6, procedures may be built upon the work of MacMillan et al. (J. Am. Chem. Soc. 2000, 122, 4243-4244), Zhang et al. (Adv. Synth. Catal. 2006, 348, 2027-2032), Puglisi et al. (Eur. J. Org. Chem. 2004, 567-573) and Selkälä et al. (Adv. Synth. Catal. 2002, 344, 941-945). In general, an alkyl ester hydrochloride of phenylalanine or tyrosine may be treated with an aqueous or organic solution of a primary amine such as, but not limited to, methylamine, n-butylamine, benzylamine or ethanolamine to give an amide hydrochloride, from which the free amine may be liberated with a suitable base such as, but not limited to, sodium hydrogen carbonate. The resultant amide may then be reacted with a ketone or aldehyde such as, but not limited to, acetone, pivalaldehyde or glyoxylic acid in an appropriate solvent to give an imidazolidin-4-one by ring closure. By acylating the phenolic alcohol in tyrosine with the appropriate acylating reagent in a suitable solvent/base-system, a monomer of general type 5 can be obtained. Optionally, by acylating a nucleophilic group in position 3 in the imidazolidin-4-one, such as an alcohol or amino group, incorporated with the help of a difunctional amine such as, but not limiting to, ethanolamine, a monomer of general type 6 can be obtained. In some cases, it may be beneficial to undertake the acylation before the fragment is incorporated into the imidazolidin-4-one, and the use of a protecting group may be necessary. Such a procedure can be exemplified by treating L-phenylalanine methyl ester hydrochloride with an excess of 8 M ethanolic methylamine for 24-48 h, followed by evaporation of volatiles to obtain L-phenylalanine-N-methylamide hydrochloride. This amide hydrochloride may be treated with excess saturated aqueous sodium hydrogen carbonate, extracted with chloroform and concentrated. Methanol and excess acetone may be added to the residue together with a catalytic amount of p-toluenesulfonic acid. The solution may be heated to reflux for 18 h, cooled to room temperature and then concentrated under reduced pressure. The residue may be taken up in diethyl ether and a solution of HCl-dioxane (4 M) is added to precipitate (5S)-5-benzyl-2,2,3-trimethylimidazolidin-4-one hydrochloride, which may be recrystallized from isopropanol. By exchanging phenylalanine with tyrosine and/or methylamine with other amines and utilizing column chromatography on silica gel with ethyl acetate/hexane or dichloromethane/methanol for purification, rather than the precipitation procedures described, other analogues suitable for acylation and preparation of monomers of the general types 5 and 6 can be prepared.

For all monomers of the general types 1-6, it is to be understood that if a linker is to be incorporated into the monomers, the synthetic sequence needs to be adjusted accordingly. It is also possible to use a (meth)acrylate as starting material that is already equipped with a linker. Several such are commercially available, such as 2-carboxyethyl acrylate, 2-carboxyethyl acrylate oligomers or 2-methacryloyloxyethyl succinic acid. In addition, for all monomers of the types 1-6, it is to be understood that certain reactions in their preparation can be made after the polymerization. The general five-membered ring of this disclosure is to be incorporated during polymerization, but certain reactions, such as a peptide coupling or minor protection can be undertaken after polymerization if appropriate.

For all monomers of the general types 1-6, the removal of a protecting group that has been utilized as part of its preparation may be undertaken after the radical polymerization. Such a deprotection protocol must be carried out according to what protecting group/groups are present. A selection of deprotection protocols is widely available in the literature already cited for the protecting group of interest. The reason for undertaking the deprotection after polymerization can be either because deprotection is more convenient after polymerization, because the protecting group provides enhanced solubility characteristics for the polymerization system of interest, or because the functionality, in unprotected, interferes with the polymerization process.

A second aspect of the current invention is to polymerize the final (meth)acrylic monomers of the general types 1-6, containing the asymmetric organocatalysts and produced according to the foregoing synthetic sequences, into polymers useful as a basis for organocatalytic reaction systems. This can be done by any of the conventional procedures for radical polymerization. The properties of the final organocatalytic system can vary greatly according to the process and nature of the physical system in which the polymerization reaction is carried out. Such systems are exemplified by bulk polymerization, solution polymerization, suspension/slurry polymerization (normal or inverse), emulsion polymerization (normal or inverse), dispersion/precipitation polymerization or seeded polymerization. The properties of the monomer must in some cases be tuned according to the polymerization system that is used. As an example, a monomer for use in suspension or emulsion polymerization with water as continuous phase cannot have a very high degree of water solubility. As such, the properties of the (meth)acrylic monomer must be tuned not only according to the characteristics of the final organocatalytic reaction system it is destined to be a part of, but also according to the characteristics of the physical polymerization system that is to be used for its polymerization. General preparatory guidelines for the radical polymerization can be readily obtained within literature of polymer chemistry. Examples of such literature found especially useful are Sourcebook of Advanced Polymer Laboratory Preparations by Sandler and Karo (Academic Press, 1998) and Polymer Synthesis: Theory and Practice by Braun et al. (4^{th} Ed., Springer, 2005).

Some special precautions for monomers of the general types 1-6 need to be undertaken, precautions that are not always readily accessible from the general literature in polymer chemistry. Especially, it has been found that monomers such as those of the general types 1-6 have a profound tendency, if not properly protected, towards reacting with peroxide radical initiators, such as benzoyl peroxide or potassium peroxodisulfate, without inducing any polymerization. The peroxides are the most widely used initiators for radical polymerization. It has been found essential for unprotected monomers to use initiators belonging to the class of the azo compounds, such as 2,2′-azobis(isobutyronitrile) (AIBN), 2,2′-azobis(isovaleronitrile) (AMBN) and 1,1′-azobis(cyclohexanecarbonitrile) as oil-soluble initiators and 2,2′-azobis(isobutyramidine hydrochloride) (AAPH) and 4,4′-azobis(cyanovaleric acid) as water-soluble initiators. The monomers may also show strong coordination to metal based catalysts/initiators, such as those used within atom transfer radical polymerization (ATRP), possibly rendering them inactive.

The radical polymerization of the (meth)acrylic monomers can be carried out with only one type of monomer or as a co-polymerization of several different monomers, either as several different (meth)acrylic monomers containing an asymmetric organocatalyst or a mixture of one or more such monomers together with one or more monomers without the organocatalyst. This co-polymerization can be used to achieve the desired characteristics of the final organocatalytic system that is of interest. It is to be understood that the polymerization can also be carried out as part of a more sophisticated physical system of polymerization, such as, but not limited to, the use of seeded polymerization. The organocatalyst-containing monomer or mixture of these together with co-polymers, crossbinders and porogens (if a macroporous polymer particle is desired), can then be used to swell the seed particles and obtain polymer particles of the microporous or macroporous type, including monodisperse ones, useful as part of an organocatalytic reaction system. Methods used for the preparation of monodisperse polymer particles by a two step swelling procedure have been disclosed by Ugelstad in U.S. Pat. No. 4,459,378 (non-magnetic) and U.S. Pat. No. 4,654,267 (magnetic), incorporated herein by reference. Further developments in the preparation of such particles can be found in WO 00/61647 (non-magnetic) and WO 2005/015216 A1 (magnetic). Alternative, more simplified procedures based on dispersion polymerization methodology and of special interest for the preparation of polymer particles for this work, microporous or macroporous, with a narrow size distribution can be found in WO 01/19885 A1, incorporated herein by reference. The (meth)acrylic monomers can also be grafted as polymer chains onto a prefabricated polymer product of the desired characteristics with the help of any radical-based polymerization technique that may suffice for the polymer product of interest. Examples of such techniques include ceric ammonium nitrate (CAN) initiated radical polymerization for particle or monolith surfaces containing proper functionalities and atom transfer radical polymerization (ATRP) for surfaces containing the appropriate group for the ATRP-system of interest or any other system that may suffice for grafting process.

The radical polymerization reactions of the current invention, of all systems covered, may also be undertaken in the presence of a chain transfer agent to control the polymer growth within the limits that is found useful for their functionalities in the finished organocatalytic system. Such a chain transfer reagent may be one of several types well known for a person skilled in the art such as, but not limited to, polyhaloalkanes (e.g. carbon tetrabromide, carbon tetrachloride and chloroform) and sulfur containing ones (e.g. 1-butanethiol, 1-dodecylthiol, 2,2′-(ethylenedioxy)diethanethiol, 2-ethylhexyl mercaptoacetate and methyl-, ethyl-, butyl- and 2-ethylhexyl 3-mercaptopropionate). The polymerization may also require more specialized additives such as stabilizers, surfactants or other additives well known for one skilled in the art and contained within the references cited for the polymerization system in question.

Embodiments of the invention are illustrated by the following non-limiting examples.

### GENERAL FOR ALL EXAMPLES

All commercially available reagents were used as received, and all solvents were used without further purification unless otherwise is noted. Inert atmosphere (N₂) is utilized only where noted specifically, and magnetic stirring is used throughout. The heating mantles used in this work were of the type Heat-On® from Radleys, either fluoropolymer coated or with anodized finish.

Thin layer chromatography (TLC) was performed on Merck silica gel 60 F₂₅₄ TLC plates, either on aluminium sheets or glass. They were visualized by UV-light, or after development in a solution of either (NH₄)₆Mo₇O₂₄.4H₂O and Ce(SO₄)₂.4H₂O in aqueous H₂SO₄, a solution of p-anisaldehyde, conc. H₂SO₄ and glacial CH₃CO₂H in 96% EtOH or a solution of KMnO₄, K₂CO₃ and NaOH in water, all followed by heating. Merck silica gel (60, 40-63 μm) was used for flash chromatography, either manually or with a Teledyne Isco CombiFlash® Companion° with PeakTrak™ software (v. 1.4.10), using EtOAc/hexanes of technical quality.

¹H NMR and ¹³C NMR spectra were recorded on a Bruker Avance™ DPX-300 or DPX-200 spectrometer operating at 300/200 MHz (¹H) or 75/50 MHz (¹³C). Chemical shifts are reported in parts per million (δ) and are, unless otherwise noted, reported relative to internal references of the solvent: 130/49.0 for CD₃OD, 2.49/39.7 for DMSO-d₆ and 7.26/77.0 for CDCl₃. For some spectra, shifts are reported relative to a residual or an added internal reference. These are given in the entry for the compound in question. Electrospray ionization mass spectra were recorded on a Micromass Q-Tof-2™ mass spectrometer. Infrared spectra were recorded on either a Nicolet Magna-IR™ 550 or a Perkin Elmer Spectrum™ One FTIR spectrometer. Melting points were determined on a Stuart® SMP3 melting point apparatus. Optical rotation was recorded using a Perkin Elmer Instruments 341 Polarimeter at room temperature. Enantiomeric excess was determined using a Thermo Scientific SpectraSYSTEM® P2000 pump with a SpectraSYSTEM® UV3000 UV/Vis detector and either a Chiralcel® OD-H, AS-H or AD-H column from Daicel Chemical Industries. The catalyst loading of the cross-linked polymer beads were all determined by CHN element analysis, calculated from the values of the nitrogen content.

### Example 1

### O-Acryloyl-trans-4-hydroxy-L-proline hydrochloride

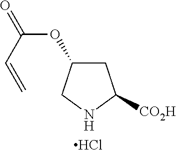

Dried and powdered trans-4-hydroxy-L-proline (12.81 g, 97.7 mmol, dried at 65° C. for 17 h) was charged into a 250 ml round-bottom glass flask and cooled in an ice/water-bath. Trifluoroacetic acid (30 ml) was added and the mixture stirred vigorously for 10 min, dissolving most of the hydroxyproline to give a viscous solution, but leaving pieces of undissolved material. Trifluoromethanesulfonic acid (1.0 ml, 11.5 mmol) was added under stirring and the reaction flask was then removed from the ice/water bath and stirred at room temperature for 10 min. Acryloyl chloride (15.8 ml, 195 mmol) was added, the reaction flask was fitted with a loose glass stopper and the reaction mixture was stirred at room temperature without any external temperature adjustment for 2 h (a clear and colourless solution with no undissolved material was obtained after ˜40 min). The reaction flask was then cooled in an ice/water-bath and diethyl ether (180 ml) was added under vigorous stirring, slowly at first. The dispersion was stirred at 0-4° C. for 10 min after completed addition and then filtered by vacuum, washed with two portions of diethyl ether and dried at room temperature for 21 h under efficient ventilation to give O-acryloyl-trans-4-hydroxy-L-proline hydrochloride (11.20 g, 52%) as a fine white powder of very good purity and suited for experiments without further purification. White crystalline powder, mp. 190-191° C. (darkens). ¹H-NMR (CD₃OD, 200 MHz): δ=6.49 (dd, J=17.2 Hz and 1.6 Hz, 1H), 6.20 (dd, J=17.2 Hz and 10.3 Hz, 1H), 5.97 (dd, J=10.3 Hz and 1.6 Hz, 1H), 5.53 (m, 1H), 4.64 (dd, J=10.5 Hz and 7.9 Hz, 1H), 3.76 (dd, J=13.3 Hz and 4.5 Hz, 1H), 3.55 (dt, J=13.3 Hz and 1.5 Hz, 1H), 2.64 (ddt, J=14.6 Hz, 7.9 Hz and 1.6 Hz, 1H), 2.48 (ddd, J=14.6 Hz, 10.5 Hz and 4.9 Hz, 1H). ¹³C-NMR (CD₃OD, 200 MHz): δ=170.5, 166.4, 133.1, 128.8, 74.1, 59.6, 52.3, 35.8. IR (KBr) cm⁻¹: 3421, 2879, 2775, 2744, 2705, 1782, 1752, 1720. HRESI-MS expected for C₈H₁₁NO₄+H, 186.0766. Found: 186.0770.

### Example 2

### Poly(O-acryloyl-trans-4-hydroxy-L-proline hydrochloride)

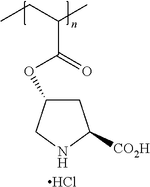

O-Acryloyl-trans-4-hydroxy-L-proline hydrochloride (1.77 g, 7.99 mmol), prepared as described in example 1, was dissolved in water (10 ml) that had been heated close to the boiling point under vigorous stirring overnight to remove oxygen. The solution was stirred at 65° C. for 15 min and flushed with nitrogen. 2,2′-Azobis(isobutyramidine hydrochloride) (AAPH, 35 mg) was added and the solution was stirred at 65-70° C. for 4 h under nitrogen and cooled to room temperature. The solution was poured into isopropanol (100 ml) and the precipitated polymer was isolated by filtration, washed with ethanol (96 vol %) and dried under vacuum over anhydrous calcium chloride for 21 h at room temperature to give poly(O-acryloyl-trans-4-hydroxy-L-proline hydrochloride) (1.48 g, 84%) as a nearly colourless solid.

### Example 3

### Poly(O-acryloyl-trans-4-hydroxy-L-proline hydrochloride)

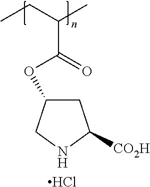

Ethanol (96 vol %) was heated to 70° C. and stirred at this temperature for 1 h to remove oxygen. O-Acryloyl-trans-4-hydroxy-L-proline hydrochloride (1.29 g, 5.82 mmol), prepared as described in example 1, was dissolved in this ethanol (10 ml) at 60-65° C. under gentle swirling (no stirring bar was added). The reaction flask was flushed with nitrogen and 2,2′-azobis(isobutyronitrile) (AIBN, 21 mg) was added. The reaction flask (without stirring) was kept at 60° C. in a bath of glycerol for 22 h under nitrogen. After cooling to room temperature, the precipitated polymer was filtered by vacuum, washed with isopropanol and dried at room temperature for 4 h and then for 19 h under vacuum over anhydrous calcium chloride at room temperature to give poly(O-acryloyl-trans-4-hydroxy-L-proline hydrochloride) (0.85 g, 66%) as a colourless solid. By using an analogous procedure with magnetic stirring and addition of a small amount of polyvinylpyrrolidone (PVP K90, M_{w}˜360 000) dispersion stabilizer, a fine white dispersion of polymer particles was obtained.

### Example 4

### N-tert-Butyloxycarbonyl-trans-4-hydroxy-L-proline

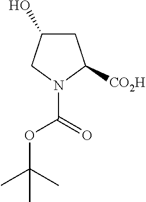

trans-4-Hydroxy-L-proline (7.60 g, 58.0 mmol) was dissolved in a solution of sodium hydroxide (2.35 g, 58.8 mmol) in water (30 ml). The solution was heated to 50° C. in a glycerol bath and a solution of di-tert-butyl dicarbonate (12.01 g, 55.0 mmol) in acetone (30 ml) was added under vigorous stirring (CO₂ evolution initiated after a couple of minutes). The reaction mixture was stirred at 50° C. for 1 hour and the acetone was evaporated in vacuo. The transparent solution was then acidified with aqueous hydrochloric acid (6 M, 10 ml), causing partial crystallization of product, and extracted with ethyl acetate (4×40 ml). The combined organic extracts were washed with brine, dried over anhydrous magnesium sulfate, filtered and evaporated in vacuo. A portion of dichloromethane was added to the residue and the solution again evaporated in vacuo to give the product as colourless foam/wax in near quantitative yield. The essentially pure product was used for the next step without further purification and data for the product was in full accordance with those reported previously in the literature (Biel et al., Chem. Eur. J. 2006, 12, 4121-4143).

### Example 5

### N-tert-Butyloxycarbonyl-O-acryloyl-trans-4-hydroxy-L-proline

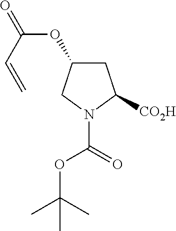

All of the N-tert-butyloxycarbonyl-trans-4-hydroxy-L-proline prepared as described in example 4 was dissolved in a mixture of dichloromethane (120 ml) and triethylamine (23.0 ml, 165 mmol). The solution was cooled to 0-5° C. in an ice/water-bath and acryloyl chloride (6.20 ml, 76.7 mmol) was added cautiously. The reaction mixture was stirred under nitrogen at 0-5° C. for 4 h. Water (60 ml) and aqueous hydrochloric acid (6 M, 8 ml) was added, the reaction mixture was stirred at 0-5° C. for another 20 min and then acidified with more aqueous hydrochloric acid (6 M, 7 ml). The organic phase was separated and washed two times with aqueous sodium hydrogen sulfate (0.3 M, 100 ml) and once with a mixture of aqueous hydrochloric acid (0.5 M, 25 ml) and brine (25 ml). Anhydrous magnesium sulfate was added together with a small portion of diatomaceous earth (Celite® 535, 1.0 g), the slurry was stirred for 10 min and filtered. Most of the solvent was evaporated in vacuo, n-butyl acetate (5 ml) was added and the rest of the dichloromethane was removed in vacuo to give a monomer solution consisting of the N-tert-butyloxycarbonyl-O-acryloyl-trans-4-hydroxy-L-proline in n-butyl acetate, used as such immediately for the next step.

For characterization, the product was purified by flash column chromatography on silica gel with ethyl acetate/hexane (3:2) to give the product as a nearly colourless oil. Data is reported for the mixture of two rotamers. ¹H-NMR (CDCl₃, 200 MHz): δ=10.50 (s, 1H), 6.37 (dd, J=17.1 Hz and 1.6 Hz, 1H), 6.05 (dd, J=17.1 Hz and 10.3 Hz, 1H), 5.82 (d, J=10.3 Hz, 1H), 5.32 (m, 1H), 4.37 (dt, J=22.1 Hz and 7.8 Hz, 1H), 3.45-3.75 (m, 2H), 2.15-2.50 (m, 2H), 1.37/1.40 (s, 9H). ¹³C-NMR (CDCl₃, 50 MHz): ó=177.5/175.7, 165.4, 155.1/153.6, 131.7, 127.8, 81.3/81.0, 72.5/72.0, 57.7/57.5, 52.2/51.8, 36.4/35.0, 28.2/28.1. IR (film) cm⁻¹: 3438, 3108, 2980, 2936, 2622, 1799, 1727, 1702. EIMS m/z (%): 184 (7), 157 (7), 112 (55), 113 (50), 68 (91), 57 (100), 56 (17), 41 (42). HRESI-MS expected for C₁₃H₁₉NO₆—H, 284.1134. Found: 284.1124.

### Example 6

### Poly(N-tert-butyloxycarbonyl-O-acryloyl-trans-4-hydroxy-L-proline)

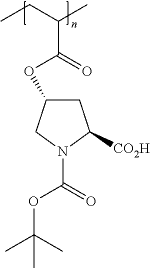

An aqueous solution (80 ml) consisting of polyvinyl alcohol (PVA, Mowiol® 40-88, M_{w}˜205 000, 87.7±1 mol % hydrolysis) and hypromellose (Methocel® K100, industrial grade, 23.0% methoxyl- and 6.5% hydroxypropyl-content, M_{w}˜26 000) suspension stabilizers (0.1% PVA and 0.1% HPMC in water) was prepared at 85° C. and allowed to reach room temperature. Citric acid monohydrate (1.0 g) was dissolved in this aqueous solution. The monomer solution from example 5 was diluted with more n-butyl acetate (15 ml) and benzoyl peroxide (231 mg, 0.95 mmol, purified by recrystallization from CHCl₃/MeOH=1:1 and dried in vacuo) was dissolved in this solution. The monomer solution was then added to the aqueous continuous phase carefully at room temperature under vigorous stirring with an ellipsoidal stirring bar so as to produce a suspension of fine droplets of monomer solution in water. The system was flushed with nitrogen while being heated to 80° C. and the suspension stirred at this temperature under nitrogen for 17 h.

The suspension was cooled to room temperature, ethanol (96 vol %, 150 ml) was added and the suspension was stirred for 15 min and filtered by vacuum (caution, filter is easily clogged). The polymer beads were suspended in methanol (200 ml) for 30 min, filtered by vacuum and washed thoroughly with water (400 ml), then with methanol (100 ml) and finally with diethyl ether (110 ml). The polymer beads were dried at room temperature for several days to give poly(N-tert-butyloxycarbonyl-O-acryloyl-trans-4-hydroxy-L-proline) as fine ivory-coloured polymer beads in the approximate general size range 60-180 μm (8.94 g, 57% overall from trans-4-hydroxy-L-proline). The product is easily soluble in trifluoroacetic or especially formic acid to give thick gels, but only slowly affected by most of the normal organic solvents. By using toluene instead of n-butyl acetate in an analogous procedure, a very similar product was obtained.

### Example 7

### Poly(O-acryloyl-trans-4-hydroxy-L-proline)

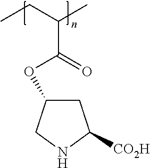

O-Acryloyl-trans-4-hydroxy-L-proline hydrochloride (2.03 g, 9.16 mmol), prepared as described in example 1, was dissolved by swirling (no stirring bar added) in water (6 ml) that had previously been heated close to the boiling point under vigorous stirring overnight to remove oxygen. The solution was flushed with nitrogen and heated to 65° C. in a bath of glycerol. 2,2′-Azobis(isobutyramidine hydrochloride) (AAPH, 36 mg) was added and dissolved by gently swirling the reaction flask. The reaction flask was kept at 65° C. under nitrogen for 3 h and then cooled with cold water. Triethylamine (0.931 g, 9.20 mmol) and water (6 ml) was added under stirring by spatula, causing the polymer to separate out of the solution and forming a cotton-like mass. The polymer was left in the aqueous solution for 30 min with occasional stirring and the entire reaction mixture was poured into methanol (100 ml). After 10 min, the polymer was separated and submerged into more methanol (50 ml), left there for 15 min with occasional stirring and finally separated and dried under vacuum over anhydrous calcium chloride for 20 h at room temperature to give poly(O-acryloyl-trans-4-hydroxy-L-proline) in near quantitative yield.

Gel permeation chromatography of a polymer sample prepared in the same manner showed a very high degree of polydispersity. The polymer in its carboxylate form (prepared by adding an equivalent of NaHCO₃ to the polymer) is active in aldol reactions of p-nitrobenzaldehyde and acetone in aqueous acetone, but was only capable of introducing a very modest enantiomeric excess of <10%. The polymer showed hardly any conversion at all in aldol reactions of p-nitrobenzaldehyde and cyclohexanone in different solvent mixtures, with very little or no stereoselectivity.

### Example 8

### O-Methacryloyl-trans-4-hydroxy-L-proline hydrochloride

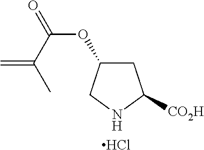

A 500 ml round bottom flask was charged with CF₃CO₂H (120 ml) and placed in an ice/water bath. Powdered trans-4-hydroxy-L-proline (32.84 g, 250 mmol, dried at 70-75° C. for 16 h) was added in small portions under vigorous stirring to give a viscous solution (leaving some small pieces of undissolved material). The reaction mixture was stirred for 5 min, then removed from the ice/water bath and CF₃SO₃H (4.0 ml, 45.8 mmol) was added. After 5 min of stirring, methacryloyl chloride (48.5 ml, 501 mmol) was added in one portion. The reaction flask was fitted with a loose glass stopper, and the reaction mixture was stirred at room temperature without any external temperature adjustment for 3 h, giving a clear and colorless solution. The reaction flask was then cooled in an ice/water bath, and Et₂O (360 ml) was added under vigorous stirring over a period of 15 min, slowly at first. The resulting white suspension was stirred at 0-5° C. for 15 min after completed addition and then filtered by vacuum. The crystals were washed with two portions of Et₂O and dried at room temperature for 23 h in a ventilated hood to give O-methacryloyl-trans-4-hydroxy-L-proline hydrochloride (38.78 g, 66%) as a fine white powder. This essentially pure material was used for the next step without further purification. The material can be recrystallized by suspending in boiling iPrOH containing a small amount of inhibitor and adding water dropwise until complete dissolution, followed by crystallization on cooling. An analytical sample of transparent and sugar-like crystals was prepared by recrystallization from boiling acetone/water in the same manner. M.p. 239-242° C. (dec.), [α]_{D}²⁰=−8.7 (c=0.138, MeOH). ¹H NMR (200 MHz, CD₃OD): δ=1.95 (s, 3H), 2.47 (ddd, 1H, J=14.5 Hz, 10.5 Hz and 4.9 Hz), 2.64 (ddt, 1H, J=14.5 Hz, 7.8 Hz and 1.6 Hz), 3.55 (dt, 1H, J=13.3 Hz and 1.5 Hz), 3.75 (dd, 1H, J=13.3 Hz and 4.7 Hz), 4.63 (dd, 1H, J=10.5 Hz and 7.8 Hz), 5.46-5.54 (m, 1H), 5.71 (quint, 1H, J=1.5 Hz), 6.21 (t, 1H, J=1.2 Hz) ppm. ¹³C NMR (50 MHz, CD₃OD): δ=18.3, 35.8, 52.3, 59.7, 74.3, 127.7, 137.0, 167.6, 170.5 ppm. IR (KBr): 3101, 2867, 1752, 1716, 1634 cm⁻¹. HRESI-MS: calcd for C₉H₁₄NO₄⁺ [M+H⁺]: 200.0922, found 200.0929.

### Example 9

### Poly(O-methacryloyl-trans-4-hydroxy-L-proline)

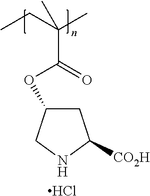

In complete analogy with example 7, a sample of poly(O-methacryloyl-trans-4-hydroxy-L-proline) was prepared by polymerization in water initiated by AAPH to give the polymeric hydrochloride, followed by liberation of the free polymer from the hydrochloride with Et₃N. The polymer is a brittle and glass-like product, less soluble than the corresponding acrylate. This example and previous ones proved that these types of acrylic and methacrylic derivatives does polymerize and can be used as a basis for cross-linked systems.

### Example 10

### O-(2-Methacryloyloxyethylsuccinoyl)-trans-4-hydroxy-L-proline hydrochloride

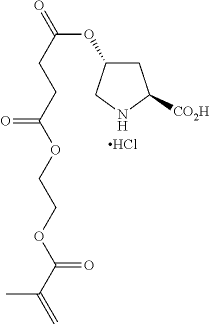

Commercial 2-methacryloyloxyethylsuccinic acid (15.0 ml, 77.5 mmol, containing 750 ppm MEHQ=4-methoxyphenol) was added to neat SOCl₂ (30.0 ml, 414 mmol) and stirred at room temperature for 30 min and at 50° C. for 1 h. The excess SOCl₂ was evaporated under reduced pressure to give 2-methacryloyloxyethylsuccinoyl chloride as a near colorless oil. A 250 ml round bottom flask was charged with CF₃CO₂H (25.0 ml), containing a spatula tip of hydroquinone, and trans-4-hydroxy-L-proline (5.134 g, 39.2 mmol) was added under vigorous stirring. The mixture was stirred for 10 min, the crude methacrylic acid chloride was added, and the reaction mixture was stirred at room temperature for 2 h to give a clear, nearly colorless solution. The solution was cooled in an ice/water bath, and Et₂O (150 ml) was added, slowly at first, under vigorous stirring. A syrupy precipitate forms, stirring was discontinued, and the precipitate was allowed to settle by gravity for 1 h. The reaction flask was removed from the ice/water bath, and the supernatant was decanted, and Et₂O (120 ml) was added. The syrupy crystals were stirred by spatula and left overnight in a refrigerator to solidify. The white solid was then broken up, vacuum-filtered and washed with Et₂O (100 ml) and dried at room temperature for 1 h to give O-(2-methacryloyloxyethylsuccinoyl)-trans-4-hydroxy-L-proline hydrochloride (10.78 g) as a white and poorly crystalline solid used immediately for the next step. M.p. 80-83° C., [α]_{D}²⁰=−10.7 (c=0.150, MeOH). ¹H NMR (CD₃OD, 200 MHz): δ=1.92 (s, 3H), 2.42 (ddd, 1H, J=14.6 Hz, 10.5 Hz and 4.6 Hz), 2.61 (dd, 1H, J=14.6 Hz and 7.8 Hz), 2.68 (s, 4H), 3.52 (d, 1H, J=13.2 Hz), 3.71 (dd, 1H, J=13.2 Hz and 4.6 Hz), 4.35 (s, 4H), 4.60 (dd, 1H, J=10.5 and 7.8 Hz), 5.45 (t, 1H, J=4.6 Hz), 5.64 (quintet, 1H, J=1.6 Hz), 6.09 (s, 1H) ppm. ¹³C NMR (CD₃OD, 50 MHz): δ=18.4, 29.6, 29.9, 35.7, 52.2, 59.5, 63.6, 63.7, 74.2, 126.6, 137.4, 168.5, 170.5, 173.1, 173.8 ppm. IR (KBr): 3383, 2931, 1758, 1736, 1716, 1636 cm⁻¹. HRMS (ESI) calcd for C₁₅H₂₂NO₈⁺ [M−Cl⁻]: 344.1345; found 344.1351.

### Example 11

### N-tert-Butyloxycarbonyl-O-(2-methacryloyloxyethylsuccinoyl)-trans-4-hydroxy-L-proline

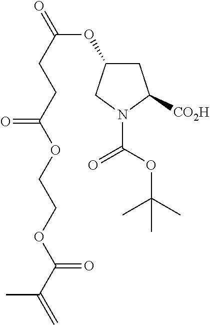

All the hydrochloride salt prepared as described in example 10 was dissolved in CH₂Cl₂ (30 ml) together with some grains of hydroquinone (˜5-10 mg) and poured into a solution of di-tert-butyl dicarbonate (6.607 g, 30.3 mmol) and Et₃N (12.0 ml) in CH₂Cl₂ (100 ml). A vigorous reaction took place, and the solution was further refluxed for 1 h and then cooled in an ice/water bath. A solution of NaHSO₄ (10.53 g, 76.3 mmol) in H₂O (100 ml) was added, the mixture was stirred for 5 min, the organic phase was separated, and the aqueous phase was extracted with CH₂Cl₂ (100 ml). The combined organic phases were washed with brine, dried over anhydrous MgSO₄ and evaporated in vacuo to give a near colorless oil of N-tert-butyloxycarbonyl-O-(2-methacryloyloxyethylsuccinoyl)-trans-4-hydroxy-L-proline in quantitative yield, used immediately for the next step. An analytical sample was prepared by flash column chromatography on silica gel with EtOAc/hexanes. Data are reported for the mixture of carbamate rotamers. Colorless oil, [α]_{D}²⁰=−31.4 (c=0.159, CHCl₃). ¹H NMR (200 MHz, CDCl₃): δ=1.40/1.44 (s, 9H), 1.92 (s, 3H), 2.15-2.50 (m, 2H), 2.61 (s, 4H), 3.49-3.72 (m, 2H), 4.20-4.50 (m, 5H), 5.28 (s, 1H), 5.57 (s, 1H), 6.10 (s, 1H), 8.29 (br. s, 1H) ppm. ¹³C NMR (50 MHz, CDCl₃): δ=18.2, 28.1, 28.7, 28.9, 34.8/36.4, 51.8/52.2, 62.2, 62.4, 72.2/72.6, 77.2, 81.0/81.5, 126.1, 135.8, 167.1, 171.6, 171.9 ppm (the two rotameric carbamate carbonyl signals were too weak for analysis). IR (film): 3107, 2980, 2935, 1739, 1640 cm⁻¹. HRMS (EST) calcd for C₂₀H₂₉NO₁₀Na⁺ [M+Na⁺]: 466.1689; found 466.1671.

### Example 12

### Crosslinked Styrenic Polymer Beads by Suspension Copolymerization

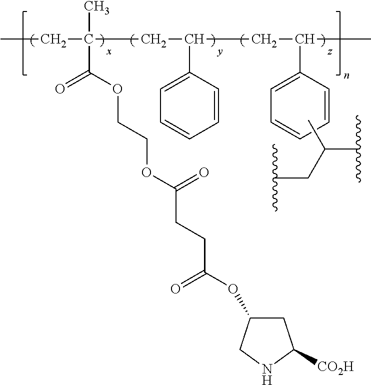

A three-necked 250 ml round bottom flask was charged with an oval magnetic stirring bar (1¼×⅝ in), potassium iodide (38 mg, inhibits polymerization in the aqueous phase), 0.3 wt % aqueous polyvinyl alcohol (Mowiol® 40-88, 150 ml) and 88% H₃PO₄ (0.20 ml). All the N-tert-butyloxycarbonyl-O-(2-methacryloyloxyethylsuccinoyl)-trans-4-hydroxy-L-proline prepared as described above was dissolved in styrene (26.04 g, 250 mmol) together with divinylbenzene (80% purity, mixture of isomers, 0.937 g, 5.76 mmol), toluene (15.0 ml) and benzoyl peroxide (343 mg, purified by recrystallization from CHCl₃/MeOH). This monomer mixture was added carefully to the aqueous solution under stirring, and the system was flushed with N₂ for 5 min. The suspension was polymerized under N₂ in a heating mantle at 85° C. for 5 h at a constant stirring rate of 600 rpm. The suspension was cooled to room temperature overnight and poured into a beaker together with water (500 ml). The beads were allowed to settle by gravity for 20 min, and the supernatant was decanted off. The process was repeated two times, and MeOH (250 ml) was added to the polymer beads, which were then stirred for a couple of minutes and filtered. The beads were washed with MeOH (250 ml), then H₂O (3000 ml) and dried under vacuum in a dessicator over P₂O₅ for 23 h at room temperature to give colorless styrenic polymer beads (30.54 g).

A portion of the beads (10.16 g) was swollen in CH₂Cl₂ (80 ml) and CF₃CO₂H (20 ml) was added. The suspension was stirred gently at room temperature for 4 h. The beads were then filtered and washed with CH₂Cl₂ (100 ml), Et₃N/MeOH (1:9, 200 ml), H₂O (250 ml) MeOH (100 ml), THF (100 ml), MeOH (100 ml) and finally H₂O (250 ml). The beads were dried at room temperature for 2 h and in a desiccator over P₂O₅ for 65 h to give a free-flowing powder (9.437 g). Elemental analysis (%): N, 0.85; C, 77.82; H, 8.38. Before testing, these beads were purified by Soxhlet-extraction for 4 h with CH₂Cl₂ (300 ml) and poured into a beaker, followed by MeOH (200 ml). After 10 min, the beads were filtered and washed with water (1000 ml) and dried at room temperature over P₂O₅ for 17 h.

### Example 13

### 2-(Hydroxy(4-nitrophenyl)methyl)cyclohexanone

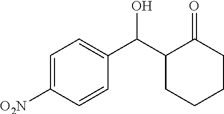

p-Nitrobenzaldehyde (60.4 mg, 0.40 mmol) was dissolved in cyclohexanone (196.3 mg, 2.0 mmol), contained in a small vial, by gentle heating on a water bath. Water (0.14 ml) was added, followed by the polymer beads (0.04 mmol, 10 mol %) prepared in example 12. The reaction was stirred gently and then left without stirring for 48 h. The reaction mixture was diluted with EtOAc and transferred to a small folded paper filter. The polymer beads were washed with additional small amounts of EtOAc (20 ml in total for dilution and washing) and the filtrate was evaporated in vacuo to yield the crude product as a yellow oil. Purification by flash column chromatography on silica with EtOAc/hexanes (gradient, 10-20% EtOAc in hexanes) yielded the product as a white solid (99.1 mg, 99%). The diastereomeric ratio (1:18.5) was determined by ¹H NMR analysis of the crude product, and the enantiomeric excess (89%) was determined by HPLC-analysis of the purified product with an AS-H chiral column (10% i-PrOH in isohexane, 1.0 ml/min, minor enantiomer Rₜ=25.6 min and major enantiomer Rₜ=27.1 min).

### Example 14

### 2-(Hydroxy(4-nitrophenyl)methyl)cyclohexanone

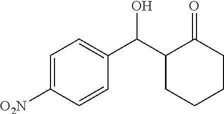

An analogous experiment to that in example 13 was carried out, but CHCl₃ (0.28 ml) was added to the reactants before the addition of water and polymer beads. The product had a diastereomeric ratio of 1:21, an enantiomeric excess of 94% and was obtained in 94% yield.

### Example 15

### 2-(Hydroxy(4-nitrophenyl)methyl)cyclohexanone

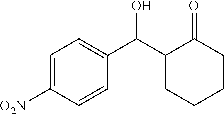

p-Nitrobenzaldehyde (604 mg, 4.0 mmol) was dissolved in a mixture of cyclohexanone (1963 mg, 20 mmol) and CHCl₃ (2.80 ml), contained in a vial, by gentle heating on a water bath. Water (1.40 ml) was added, followed by the polymer beads (0.40 mmol, 10 mol %) prepared in example 12. The reaction was stirred gently and then left without stirring for 24 h. The reaction mixture was diluted with EtOAc and transferred to a Büchner-funnel. The polymer beads were washed with additional small amounts of EtOAc (50 ml in total for dilution and washing) and the filtrate was evaporated in vacuo to yield the crude product as a yellow oil. Purification by flash column chromatography on silica with EtOAc/hexanes (gradient, 10-20% EtOAc in hexanes) yielded the product as a white solid (857.6 mg, 86%) with a diastereomeric ratio of 1:22 and an enantiomeric excess of 91%. The polymer beads were washed with several portions of CH₂Cl₂ and dried at room temperature for at least 24 h. The polymer beads (0.36 mmol, 10 mol %) were then reused without further purification in a completely analogous experiment, using p-nitrobenzaldehyde (544 mg, 3.6 mmol), cyclohexanone (1766 mg, 18.0 mmol), CHCl₃ (2.55 ml) and water (1.27 ml) to a give the product in 90% yield with a diastereomeric ratio of 1:40 and an enantiomeric excess of 97%.

### Example 16

### Cross-Linked Styrenic Beads Containing Prolineamide

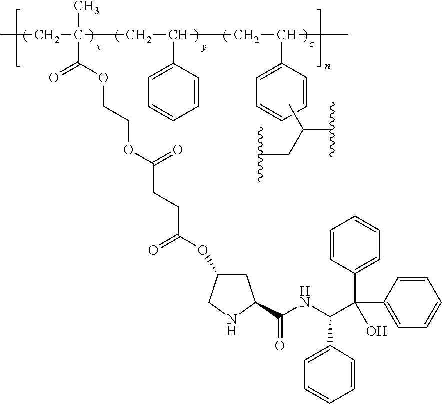

The rest of the protected (Boc-containing) beads, prepared in example 12 (˜20 g), were swollen in CH₂Cl₂, loaded into a cellulose thimble (43×123 mm) and extracted for 6 h with CH₂Cl₂ (350 ml) in a Soxhlet-extractor. The beads were transferred to a beaker, and MeOH (250 ml) was added. After 15 min, the beads were filtered and dried at room temperature for 48 h.

A portion of these purified beads (1.00 g) was swollen in CH₂Cl₂ (20 ml), and iPr₂NEt (0.2630 g, 2.03 mmol) was added. Under very gentle stirring, (S)-2-amino-1,1,2-triphenylethanol (0.6073 g, 2.10 mmol) and O-(Benzotriazol-1-yl)-N,N,N′,N′-tetramethyluronium hexafluorophosphate (HBTU, 0.7964 g, 2.10 mmol) were added, and the mixture stirred gently at room temperature for 2 h. The beads were filtered, washed with a small amount of CH₂Cl₂, deprotected with CF₃CO₂H/CH₂Cl₂ (1:4), purified by Soxhlet-extraction with CH₂Cl₂ and dried at room temperature to give prolineamide-containing beads.

### Example 17

### 2-(Hydroxy(4-nitrophenyl)methyl)cyclohexanone

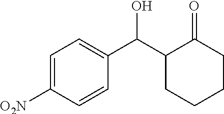

A completely analogous experiment to that in example 13 was carried out using the polymer beads prepared in example 16. The product had an enantiomeric excess of 96% and was obtained in 89% yield.

### Example 18

### 2-(Hydroxy(4-nitrophenyl)methyl)cyclohexanone

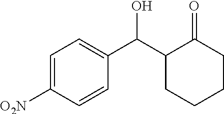

An analogous experiment to that in example 13 was carried out using the polymer beads prepared in example 16, but CHCl₃ (0.28 ml) was added to the reactants before the addition of water and polymer beads. The product had an enantiomeric excess of 98% and was obtained in 85% yield.

### Example 19

### N-tert-Butyloxycarbonyl-O-methacryloyl-trans-4-hydroxy-L-proline

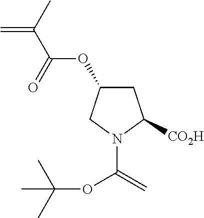

A solution of di-tert-butyl dicarbonate (5.452 g, 25.0 mmol) and Et₃N (10.0 ml, 71.7 mmol) in CH₂Cl₂ (50 ml) containing a few grains of hydroquinone (˜1 mg) was prepared, and O-methacryloyl-trans-4-hydroxy-L-proline hydrochloride (6.180 g, 26.2 mmol) was added in small portions over a period of 10 min by a powder funnel. After completed addition, more CH₂Cl₂ (40 ml) was added to flush down traces of crystals. The suspension was refluxed for 30 min, giving a clear and colorless solution. The solution was cooled in an ice/water bath and a solution of NaHSO₄ (8.05 g) in H₂O (70 ml) was added under stirring. After 5 min, the phases were separated and the aqueous phase extracted with CH₂Cl₂ (70 ml), the combined organic phases were washed with a small amount of brine, dried over anhydrous MgSO₄ and evaporated in vacuo to give a colorless oil of N-tert-butyloxycarbonyl-O-methacryloyl-trans-4-hydroxy-L-proline in virtually quantitative yield, pure except for residual tent-butanol and used directly for the next step. An analytical sample was prepared by flash column chromatography on silica gel with EtOAc/hexanes. Data reported for the mixture of carbamate rotamers. Colorless oil, [α]_{D}²⁰=−48.3 (c=0.145, CHCl₃). ¹H NMR (200 MHz, CDCl₃): δ=1.42/1.45 (s, 9H), 1.92 (s, 3H), 2.21-2.56 (m, 2H), 3.54-3.80 (m, 2H), 4.36/4.48 (t, 1H, J=7.8 Hz), 5.34 (br. s, 1H), 5.59 (s, 1H), 6.09 (s, 1H), 8.97 (br. s, 1H) ppm. ¹³C NMR (50 MHz, CDCl₃): δ=18.1, 28.2/28.3, 34.9/36.5, 51.9/52.3, 57.6/57.8, 72.1/72.5, 81.1/81.6, 126.4, 135.8, 153.7/155.5, 166.6, 175.6, 178.0 ppm. IR (film): 3107, 2980, 2934, 1750, 1716 cm⁻¹.

### Example 20

### Cross-Linked Methacrylic Polymer Beads by Suspension Copolymerization

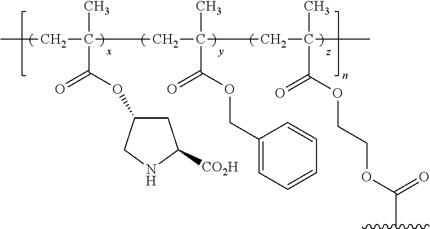

A three-necked 250 ml round bottom flask was charged with an oval magnetic stirring bar (1¼×⅝ in), potassium iodide (33 mg, inhibits polymerization in the aqueous phase), 0.3 wt % aqueous polyvinyl alcohol (Mowiol® 40-88, 150 ml) and 88% H₃PO₄ (0.40 ml). All the N-tert-butyloxycarbonyl-O-methacryloyl-trans-4-hydroxy-L-proline prepared as described in example 14 was dissolved in benzyl methacrylate (30.85 g, 175 mmol) together with ethyleneglycol dimethacrylate (90% purity, 0.923 g, 4.19 mmol), toluene (20.0 ml) and benzoyl peroxide (365 mg, purified by recrystallization from CHCl₃/MeOH). This monomer mixture was added carefully to the aqueous solution under stirring, and the system was flushed with N₂ for 5 min. The suspension was polymerized under N₂ in a heating mantle at 80° C. for 5 h at a constant stirring rate of 600 rpm.

The suspension was cooled to room temperature and poured into a beaker together with water (500 ml). The beads were allowed to settle by gravity for 10 min, and the supernatant was decanted off. The process was repeated once more, and the polymer beads were then filtered, washed with water (800 ml) and MeOH (300 ml) and dried at room temperature to give colorless methacrylic polymer beads (39.80 g) in the general size range 20-150 μm. Elemental analysis (%): N, 0.85; C, 71.77; H, 7.33.

A portion of the beads (32.18 g) was swollen in CH₂Cl₂ (200 ml) and CF₃CO₂H (50 ml) was added. The suspension was left at room temperature for 4 h with occasional stirring. The beads were then filtered and washed with CH₂Cl₂ (200 ml), Et₃N/MeOH (1:9, 250 ml), Et₃N/THF (1:9, 100 ml), THF (100 ml), MeOH (200 ml), H₂O (500 ml), MeOH (200 ml) and finally H₂O (500 ml). The beads were dried at room temperature for 14 h and in a desiccator over P₂O₅ for 71 h to give a free-flowing powder (29.98 g). Elemental analysis (%): N, 1.05; C, 70.48; H, 7.35. Before testing, a portion of these beads (˜20 g) were purified by Soxhlet-extraction for 6 h with CH₂Cl₂ (300 ml) and poured into a beaker, followed by MeOH (250 ml). After 10 min, the beads were filtered and washed with water (1000 ml) and dried at room temperature over P₂O₅ for 18 h.

### Example 21

### 2-(Hydroxy(4-nitrophenyl)methyl)cyclohexanone

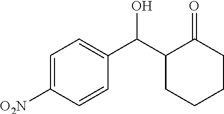

A completely analogous experiment to that in example 13 was carried out using the polymer beads prepared in example 20. The product had a diastereomeric ratio of 1:12, an enantiomeric excess of 91% and was obtained in 99% yield.

### Example 22

### 2-(Hydroxy(4-nitrophenyl)methyl)cyclohexanone

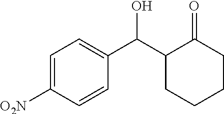

An analogous experiment to that in example 13 was carried out using the polymer beads prepared in example 20, but CHCl₃ (0.28 ml) was added to the reactants before the addition of water and polymer beads. The product had a diastereomeric ratio of 1:10, an enantiomeric excess of 93% and was obtained in 77% yield.

### Example 23

### Cross-Linked Polymer Support by Dispersion Copolymerization

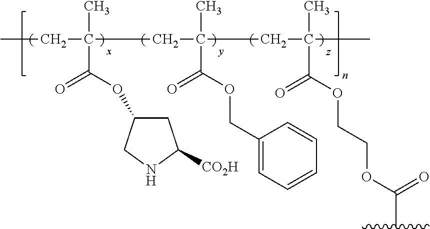

A three-necked 250 ml round bottom flask was charged with an oval magnetic stirring bar (1¼×⅝ in), O-methacryloyl-trans-4-hydroxy-L-proline hydrochloride (3.026 g, 12.8 mmol), benzyl methacrylate (18.11 g, 103 mmol), ethyleneglycol dimethacrylate (90% purity, 0.530 g, 2.41 mmol), AIBN (276 mg) and a solution of polyvinylpyrrolidone (PVP K90, 72 mg) in MeOH (180 ml). The system was flushed with N₂ for 5 min and polymerized under N₂ in a Radleys Heat-On® heating mantle at 60° C. for 5 h at a constant stirring rate of 600 rpm. The agglomerated dispersion was diluted with MeOH (100 ml), filtered and washed with MeOH (100 ml) to give the polymer as a fluffy powder. The powder was transferred to a beaker and CH₂Cl₂ (250 ml), H₂O (50 ml) and Et₃N (15 ml) were added. The mixture was stirred by spatula for 10 min to give a homogeneous gel. The gel was left at room temperature for 30 min with occasional stirring and then filtered and washed with MeOH (200 ml), THF (100 ml), MeOH (200 ml) and finally THF (100 ml). The polymer was removed from the filter while moist with THF, divided into a fine granulate with a metal spoon (while moist with THF) and dried at room temperature for 40 h to give the polymer support as a convenient white granulate (15.97 g). Elemental analysis (%): N, 0.27; C, 73.70; H, 7.21. Electron microscopy revealed that the polymer support consists of agglomerated irregular polymer beads.

### Example 24

### 2-(Hydroxy(4-nitrophenyl)methyl)cyclohexanone

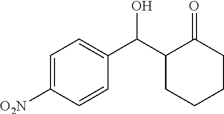

A completely analogous experiment to that in example 13 was carried out using the polymer product prepared in example 23. The product had a diastereomeric ratio of 1:15, an enantiomeric excess of 94% and was obtained in 78% yield.

### Example 25

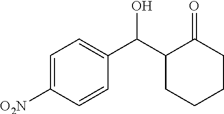

An analogous experiment to that in example 13 was carried out using the polymer product prepared in example 23, but CHCl₃ (0.28 ml) was added to the reactants before the addition of water and polymer beads. The product had a diastereomeric ratio of 1:6.5, an enantiomeric excess of 99% and was obtained in 81% yield.

### Example 26

### trans-4-Hydroxy-α,α-diphenyl-L-prolinol hydrochloride

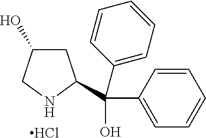

To a stirred suspension of trans-4-hydroxy-L-proline (55.38 g, 422 mmol) in EtOH (96%, 500 ml), cooled in an ice/water bath, was added SOCl₂ (46.0 ml, 634 mmol) via an additional funnel over a period of 15 min. The suspension was then heated to reflux and kept there for 3 h. The resultant clear and colorless solution was cooled in an ice/water bath, and the product crystallized. A portion of Et₂O (500 ml) was added, and the suspension was stirred vigorously, vacuum-filtered and washed with Et₂O (100 ml). The product was dried at room temperature for 23 h to give trans-4-hydroxy-L-proline ethyl ester hydrochloride (73.12 g, 88%) as white fibrous crystals. A three-necked 2000 ml round bottom flask equipped with a long reflux condenser, addition funnel and glass stopper was charged with an oval stirring bar (50×20 mm) and Mg-turnings (40.24 g, 1655 mmol). Dry Et₂O (80 ml) was added to the Mg-turnings, followed by a small portion of a solution of bromobenzene (175 ml, 1665 mmol) in dry Et₂O (500 ml) to initiate the reaction. The rest of the PhBr-solution was then added over a period of 1 h 40 min, keeping the addition rate so as to maintain gentle reflux. After addition, stirring was continued for 1 h to dissolve most Mg and the mixture was diluted with dry Et₂O (420 ml) and cooled in an ice/water bath. trans-4-Hydroxy-L-proline ethyl ester hydrochloride (46.46 g, 237 mmol) was added under stirring, the reaction flask was transferred to a heating mantle and refluxed for 5 h under stirring, giving a quite clear solution and precipitate. The reaction flask was then cooled in an ice/water bath, and its contents were carefully poured into crushed ice (1250 ml) contained in a 3000 ml beaker. Concentrated aqueous HCl (37%, 140 ml) and water (600 ml) was added under stirring by a glass rod. As much as possible of the colored top organic layer was decanted, Et₂O (200 ml) was added and then decanted off after stirring. Concentrated aqueous NH₃ (25%, 25 ml) was added to adjust pH of the slurry to approximately 9, and the slurry was vacuum-filtered. The filter cake was washed with water (1000 ml) and MTBE (methyl tert-butyl ether, 200 ml), and transferred to a 600 ml beaker. The crude product was suspended in MeOH (250 ml), and CF₃CO₂H (19 ml, 247 mmol) was added to dissolve the material to give a dark-colored, but clear solution which was vacuum-filtered to remove residual Mg. An ice-cold methanolic HCl solution (prepared by dropping 25 ml of acetyl chloride into 100 ml of MeOH under cooling from an ice/water bath) was added, followed by Et₂O (700 ml). A precipitate quickly formed, the suspension was stirred vigorously for 10 min, then vacuum-filtered, and the product washed with Et₂O (350 ml). A second portion of material precipitated from the mother liqueur on standing and was isolated (after cooling in an ice/water bath) in the same manner and dried for 24 h at room temperature to give trans-4-hydroxy-α,α-diphenyl-L-prolinol hydrochloride (32.10 g, 44% in total) as near colorless and fibrous material of very good purity, used as is for the next step. An analytical sample was prepared by recrystallization of this material from 96% EtOH. M.p. 266-268° C. (dec.), [α]_{D}²⁰=+8.6 (c=0.232, MeOH). ¹H NMR (200 MHz, CD₃OD, calibrated by residual EtOH at δ=1.17): δ=1.95 (dd, 1H, J=13.7 Hz and 6.8 Hz), 2.19 (ddd, 1H, J=13.7 Hz, 10.7 Hz and 4.1 Hz), 3.22 (d, 1H, J=12.2 Hz), 3.34 (dd, 1H, J=12.2 Hz and 14 Hz), 4.53 (br. s, 1H), 5.07 (dd, 1H, J=10.7 Hz and 6.8 Hz), 7.18-7.45 (m, 6H), 7.47-7.54 (m, 2H), 7.62-7.70 (m, 2H) ppm. ¹³C NMR (50 MHz, CD₃OD): δ=36.4, 55.4, 66.6, 70.7, 78.2, 126.7, 126.8, 128.6, 128.8, 129.5, 129.9, 145.3, 145.4 ppm. IR (KBr): 3400, 3306, 3027, 1450, 991 cm⁻¹. HRMS (ESI) calcd for C₁₇H₂₀NO₂⁺ [M−Cl⁻]: 270.1494; found 270.1491.

### Example 27

### trans-4-Hydroxy-α,α-diphenyl-L-prolinol

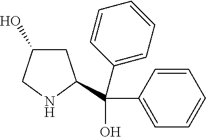

This compound, the free amine of the product in example 26, was prepared the same way as for the hydrochloride in example 26, except that the crude product was directly recrystallized from a MeOH/PhMe/THF-mixture, instead of the CF₃CO₂H/HCl-treatment.

M.p. 190-192° C., [α]_{D}²⁰=−115.3 (c=0.215, DMSO). ¹H NMR (200 MHz, DMSO-d₆): δ=1.17-1.31 (m, 1H), 1.50-1.67 (m, 1H), 2.37 (s, 1H), 2.72 (dd, 1H, J=10.9 Hz and 1.4 Hz), 2.94 (dd, 1H, J=10.9 Hz and 4.4 Hz), 4.08 (s, 1H), 4.44-4.56 (m, 2H), 5.05 (s, 1H), 7.06-7.31 (m, 6H), 7.40-7.47 (m, 2H), 7.54-7.62 (m, 2H) ppm. ¹³C NMR (50 MHz, DMSO-d₆): δ=36.7, 55.6, 63.0, 71.2, 77.5, 125.5, 126.1, 126.2, 126.7, 127.9, 146.8, 148.3 ppm. IR (KBr): 3301, 3281, 3087, 3059, 2977, 1495, 1446 cm⁻¹. HRMS (ESI) calcd for C₁₇H₂₀NO₂⁺ [M+H⁺]: 270.1494; found 270.1488.

### Example 28

### O-(2-Methacryloyloxyethylsuccinoyl)-trans-4-hydroxy-α,α-diphenyl-L-prolinol hydrochloride

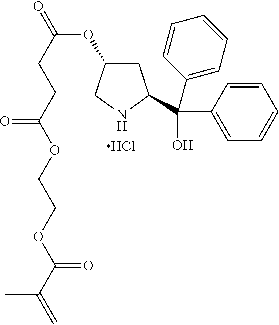

A 250 ml round bottom flask was charged with commercial 2-methacryloyloxyethylsuccinic acid (48.54 g, 211 mmol, containing 750 ppm MEHQ), and SOCl₂ (75.0 ml, 1034 mmol) was added. The reaction mixture was stirred at room temperature for 30 min, MEHQ (25 mg) was added, and stirring was continued at 50° C. for 30 min. Excess SOCl₂ was removed in vacuo to give the 2-methacryloyloxyethylsuccinoyl chloride as a light yellow oil. A 500 ml round bottom flask was charged with trans-4-hydroxy-α,α-diphenyl-L-prolinol hydrochloride (32.00 g, 105 mmol), which was then dissolved by addition of CF₃CO₂H (100 ml). The solution was cooled in an ice/water bath, and the crude methacrylic acid chloride was added. The light brown reaction mixture was removed from the ice/water bath and was stirred at room temperature for 2 h. It was then cooled again in an ice/water bath and carefully diluted with Et₂O (500 ml). The resulting dispersion was stirred vigorously for 20 min, then removed from the ice/water bath and vacuum-filtered. The solid was washed with Et₂O (300 ml) and dried at room temperature overnight. The crude product was transferred to a 500 ml beaker together with hydroquinone (120 mg) and dissolved in EtOH (96 vol %, 300 ml) under stirring by heating to the boiling point. Boiling MTBE (200 ml) was added slowly to the colored solution. Crystallization initiated, stirring was discontinued, and the solution left for crystallization at room temperature for 5 h. The crystals were vacuum-filtered, washed with MTBE (300 ml) and dried at room temperature for 65 h to give O-(2-methacryloyloxyethylsuccinoyl)-trans-4-hydroxy-α,α-diphenyl-L-prolinol hydrochloride as a white and fluffy solid (37.78 g, 70%). M.p. 187-190° C. (dec.), [α]_{D}²⁰=−87.7 (c=0.195, CHCl₃). ¹H NMR (200 MHz, DMSO-d₆): δ=1.60-1.75 (m, 1H), 1.85 (s, 3H), 2.21-2.40 (m, 1H), 2.63 (s, 4H), 3.23-3.56 (m, 2H), 4.28 (s, 4H), 5.01 (br. s, 1H), 5.24 (s, 1H), 5.66 (s, 1H), 6.02 (s, 1H), 6.69 (s, 1H), 7.12-7.42 (m, 6H), 7.43-7.54 (m, 2H), 7.65-7.78 (m, 2H), 8.99 (br. s, 1H), 10.51 (br. s, 1H) ppm.

¹³C NMR (50 MHz, DMSO-d₆): δ=18.1, 28.7, 29.1, 32.6, 51.2, 62.2, 62.6, 64.0, 73.1, 77.1, 125.4, 126.3 (2×), 127.2, 127.5, 128.4, 128.6, 135.8, 144.4, 144.6, 166.6, 171.6, 172.1 ppm. IR (KBr): 3235, 2960, 1733, 1636, 1169, 1149 cm⁻¹. HRMS (ESI) calcd for C₂₇H₃₂NO₇⁺ [M−Cl⁻]: 482.2178; found 482.2163.

### Example 29

### O-(2-Methacryloyloxyethylsuccinoyl)-trans-4-hydroxy-α,α-diphenyl-L-prolinol trimethylsilyl ether

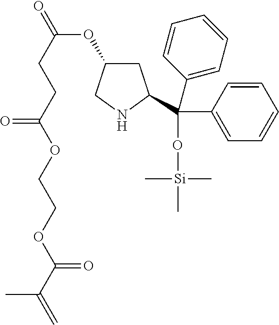

O-(2-Methacryloyloxyethylsuccinoyl)-trans-4-hydroxy-α,α-diphenyl-L-prolinol hydrochloride (5.2975 g, 10.2 mmol) was suspended in CH₂Cl₂ (40 ml), and aqueous K₂CO₃ (10 wt %, 40 ml) was added. The mixture was stirred vigorously for 5 min and separated. The aqueous phase was extracted with CH₂Cl₂ (20 ml) and the combined organic phases were dried over anhydrous MgSO₄ and filtered into a round bottom flask. The MgSO₄ was washed with extra CH₂Cl₂ (20 ml) and filtered into the same flask. Iodine (0.0410 g, 0.16 mmol) and hexamethyldisilazane (3.20 ml, 15.3 mmol) was added to the clear solution and the reaction mixture was stirred at room temperature for 4 h and quenched by addition of MeOH (3 ml). After stirring for 10 min, the volatiles were evaporated in vacuo and the residual oil was dissolved in CH₂Cl₂ (40 ml) and treated with a solution of Na₂S₂O₃.5H₂O (4.52 g, 18.2 mmol) in water (40 ml) under stirring for 5 min. The mixture was separated, and the organic phase was dried over anhydrous MgSO₄, filtered and evaporated in vacuo to give O-(2-methacryloyloxyethylsuccinoyl)-trans-4-hydroxy-α,α-diphenyl-L-prolinol trimethylsilyl ether as a clear and only slightly colored oil of good purity, and used as is for the polymerization. An analytical sample was prepared by flash column chromatography on silica gel with EtOAc/hexanes. Colorless oil, [α]_{D}²⁰=−8.5 (c=0.106, CHCl₃). ¹H NMR (CD₃OD, 200 MHz): δ=−0.10 (s, 9H), 1.73 (dd, 1H, J=14.2 Hz and 7.0 Hz), 1.84-1.98 (m, 4H), 2.60 (br. s, 4H), 2.75 (dd, 1H, J=12.2 Hz and 4.7 Hz), 2.86 (d, 1H, J=12.2 Hz), 4.28-4.43 (m, 5H), 4.85-4.93 (m, 1H), 5.60 (br. s, 1H), 6.08 (s, 1H), 7.15-7.50 (m, 10H) ppm. ¹³C NMR (50 MHz, CD₃OD): δ=2.3, 18.4, 29.8, 30.1, 35.6, 53.5, 63.5, 633, 64.9, 76.9, 84.2, 126.6, 128.2, 128.3, 128.7, 128.8, 128.9, 129.5, 137.4, 146.2, 147.2, 168.4, 173.6, 173.7 ppm. HRMS (ESI) calcd for C₃₀H₄₀NO₇Si⁺ [M+H⁺]: 554.2574; found 554.2563.

### Example 30

### Cross-Linked Methacrylic Beads Containing Diarylprolinol Trimethylsilyl Ether by Suspension Copolymerization

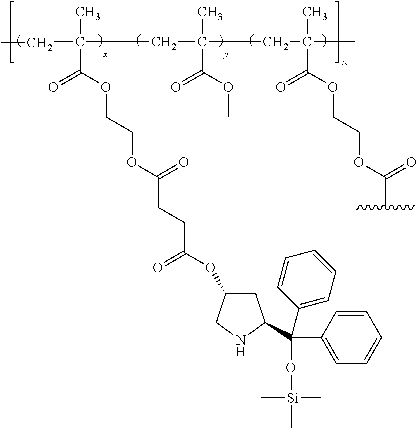

A three-necked 250 ml round bottom flask was charged with an egg-shaped magnetic stirring bar (1½×⅝ in), potassium iodide (60 mg, inhibits polymerization in the aqueous phase), K₂CO₃ (185 mg), 0.5 wt % aqueous polyvinyl alcohol (Mowiol® 40-88, 130 ml). A mixture of all the O-(2-methacryloyloxyethylsuccinoyl)-trans-4-hydroxy-α,α-diphenyl-L-prolinol trimethylsilyl ether prepared in example 29 was dissolved in methyl methacrylate (16.55 g, 165 mmol) together with ethyleneglycol dimethacrylate (0.712 g, 3.59 mmol), toluene (20 ml) and 2,2′-Azobis(2-methylbutyronitrile) (222 mg). This monomer mixture was added carefully to the aqueous solution under stirring, and the system was flushed with N₂ for 5 min. The suspension was polymerized under N₂ in a heating mantle at 70° C. for 16 h at a constant stirring rate of 550 rpm.

The suspension was allowed cool and poured into a beaker containing MeOH (300 ml). The beads were allowed to settle by gravity, and the supernatant was decanted off. The process was repeated once more after addition of MeOH (300 ml), the beads were slurried in water, vacuum-filtered and washed with water (1500 ml). The beads were purified by Soxhlet-extraction with CH₂Cl₂ to give nearly colorless methacrylic polymer beads containing diarylprolinol trimethylsilyl ether.

1. A chiral polymer organocatalyst comprising a main chain and side chain organocatalytic groups covalently attached to the main chain, which organocatalytic groups comprise an amino acid or amino acid derivative of the following general formula, in which one stereoisomeric form predominates: 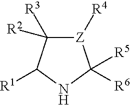
wherein the catalyst is bound to the polymer main chain via R¹, R², R⁴, R⁵ or R⁶ through a linker (L) or direct bond, and wherein R¹-R⁶ and Z are defined as follows:
Z is CH or N;
R¹ is H, a naturally occurring alpha-amino acid side chain or a non-natural commercially available alpha-amino acid side chain that may contain L;
R² is H, O (doubly bonded to give a carbonyl), O-L (where L is a linker), NH-L or L;
R³ is H or doubly bonded to give a carbonyl with R² when R² is O;
R⁴ is H, C₁-C₆ alkyl or L
R⁵ is H, CO₂H, C₁-C₆ alkyl, benzyl, L, CONHR (in which R is alkyl, aryl, heteroaryl, arylalkyl or, heteroarylalkyl), tetrazolyl, CH₂ coupled to a triazole moiety, an esterified CH₂OH or CO₂R (in which R is alkyl, aryl, heteroaryl, arylalkyl or heteroarylalkyl), 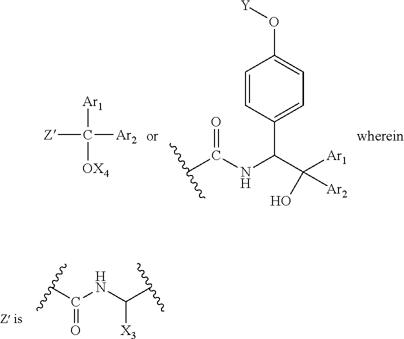
or a direct bond,
X₄ is H, Me₃Si or Et₃Si, X₃ comprises a naturally-occurring alpha-amino acid side chain, H, C₁-C₆ alkyl or phenyl, Ar₁ and Ar₂ are each independently aryl or heteroaryl, and Y denotes the position of attachment to the main chain or linker; and
R⁶ is H, CO₂H, C₁-C₆ alkyl, benzyl or L; and
wherein the polymer organocatalyst comprises a cross-linked polymer.

2. A polymer organocatalyst according to claim 1, wherein Z is CH and R² is attached to the main chain, optionally via a linker.

3. A polymer organocatalyst according to claim 2, wherein R⁵ is CO₂H and R¹, R⁴ and R⁶ are each H.

4. A polymer organocatalyst according to claim 2, wherein R⁵ is 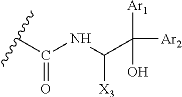
and R¹, R³, R⁴ and R⁶ are each H.

5. A polymer organocatalyst according to claim 2, wherein R⁵ is 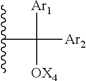
and R¹, R³, R⁴ and R⁶ are each H.

6. A polymer organocatalyst according to claim 1, wherein Z is CH, R⁵ comprises 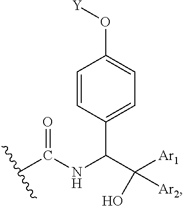
and R¹, R², R³, R⁴ and R⁶ are each H.

7. A polymer organocatalyst according to claim 1, wherein Z is N, R² and R³ together form carbonyl, R¹ is attached to the main chain, optionally by a linker, R⁴ is C₁-C₆ alkyl and R⁵ and R⁶ are each independently C₁-C₆ alkyl, benzyl or carboxylate.

8. A polymer organocatalyst according to claim 1, wherein Z is N, R² and R³ together form carbonyl, R⁴ is attached to the main chain, optionally by a linker, R⁵ and R⁶ are each independently C₁-C₆ alkyl, benzyl or carboxylate and R¹ is Ar₁—CH₂.

9. A polymer organocatalyst according to claim 1, wherein each amino acid or amino acid derivative is attached to the main chain via a linker which comprises a linear or branched hydrocarbylene and which has a chain length in the range of from 2 to 25 atoms.

10. A polymer organocatalyst according to claim 1, wherein each amino acid or amino acid derivative is attached to the main chain via a linker which comprises an ethyl succinoyl linker.

11. A polymer organocatalyst according to claim 1, wherein the main chain polymer comprises a polyacrylate or polymethylacrylate.

12. A polymer organocatalyst according to claim 1, wherein the main chain polymer comprises a copolymer.

13. A polymer organocatalyst according to claim 12, wherein the copolymer includes bi- or higher order functional monomer units which provide a cross-linked structure.

14. A polymer organocatalyst according to claim 1, which is in the form of polymer particles.

15. A process for the preparation of a chiral polymer organocatalyst, which process comprises:
providing monomers comprising an organocatalytic group covalently attached to a polymerisable unit; and polymerising the polymerisable units to form the polymer organocatalyst; wherein the organocatalytic group comprises 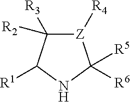
wherein the catalyst is bound to the polymer main chain via R¹, R², R⁴, R⁵ or R⁶ through a linker (L) or direct bond, and wherein R¹-R⁶ and Z are defined as follows:
Z is CH or N;
R¹ is H, a naturally occurring alpha-amino acid side chain or a non-natural commercially available alpha-amino acid side chain that may contain L;
R² is H, O (doubly bonded to give a carbonyl), O-L (where L is a linker), NH-L or L;
R³ is H or doubly bonded to give a carbonyl with R² when R² is O;
R⁴ is H, C₁-C₆ alkyl or L
R⁵ is H, CO₂H, C₁-C₆ alkyl, benzyl, L, CONHR (in which R is alkyl, aryl, heteroaryl, arylalkyl or, heteroarylalkyl), tetrazolyl, CH₂ coupled to a triazole moiety, an esterified CH₂OH or CO₂R (in which R is alkyl, aryl, heteroaryl, arylalkyl or heteroarylalkyl) 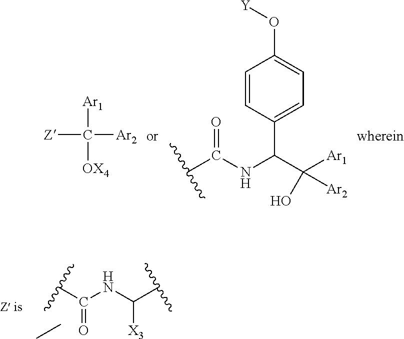
or a direct bond,
X₄ is H, Me₃Si or Et₃Si, X₃ comprises a naturally-occurring alpha-amino acid side chain, H, C₁-C₆ alkyl or phenyl, Ar₁ and Ar₂ are each independently aryl or heteroaryl, and Y denotes the position of attachment to the main chain or linker; and
R⁶ is H, CO₂H, C₁-C₆ alkyl, benzyl or L; and
wherein the step of polymerising includes cross-linking polymer main chains.

16. A process according to claim 15, wherein Z is CH and R² is attached to the main chain, optionally via a linker.

17. A process according to claim 16, wherein R⁵ is CO₂H and R¹, R⁴ and R⁶ are each H.

18. A process according to claim 16, wherein R⁵ is 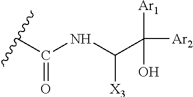
and R¹, R³, R⁴ and R⁶ are each H.

19. A process according to claim 16, wherein R⁵ is 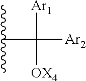
and R¹, R³, R⁴ and R⁶ are each H.

20. A process according to claim 15, wherein Z is CH, R⁵ comprises 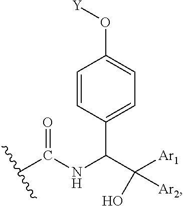
and R¹, R², R³, R⁴ and R⁶ are each H.

21. A process according to claim 15, wherein Z is N, R² and R³ together form carbonyl, R¹ is attached to the main chain, optionally by a linker, R⁴ is C₁-C₆ alkyl and R⁵ and R⁶ are each independently C₁-C₆ alkyl, benzyl or carboxylate.

22. A process according to claim 15, wherein Z is N, R² and R³ together form carbonyl, R⁴ is attached to the main chain, optionally by a linker, R⁵ and R⁶ are each independently C₁-C₆ alkyl, benzyl or carboxylate and R¹ is Ar₁—CH₂.

23. A process according to claim 15, wherein each amino acid or amino acid derivative is attached to the polymerisable unit via a linker which comprises a linear or branched hydrocarbylene and which has a chain length in the range of from 2 to 25 atoms.

24. A process according to claim 15, wherein each amino acid or amino acid derivative is attached to the main chain via a linker which comprises an ethyl succinoyl linker.

25. A process according to claim 15, wherein the polymerisable unit comprises an acrylate or methylacrylate.

26. A process according to claim 15, which further comprises providing a co-monomer, wherein the step of polymerising the polymerisable units comprises copolymerising the polymerisable unit with the co-monomer.

27. A process according to claim 26, wherein the comonomer comprises a bi- or higher order functional monomer for providing cross-linking in the polymer.

28. A process according to claim 15, wherein the monomer comprises any one of the following: 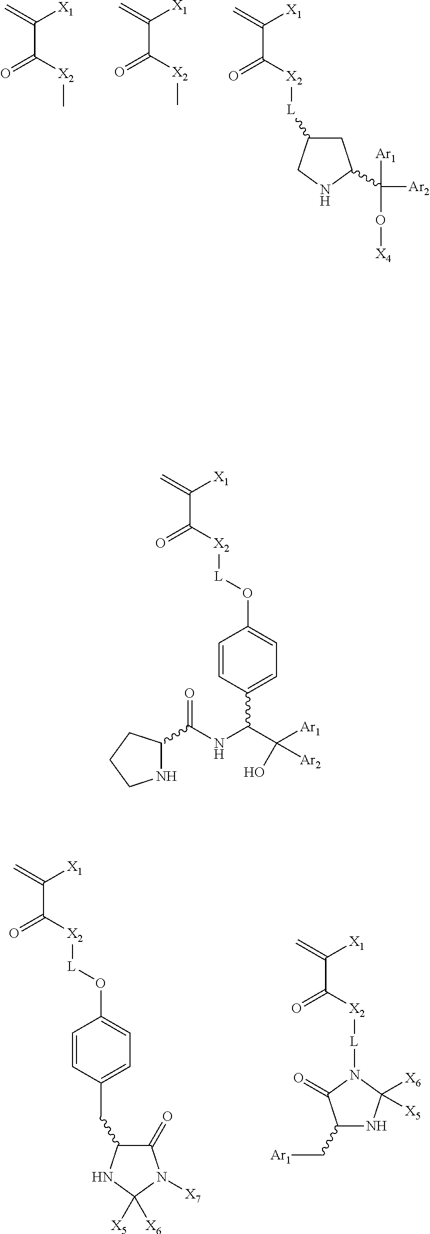
in which L represents the linker, X₁ is H or Me, X₂ is O or NH, X₅ and X₆ are each independently C₁-C₆ alkyl, benzyl or carboxylate and X₇ is C₁-C₆ alkyl.

29. A process according to claim 15, wherein the step of polymerising forms polymer particles.

30. A process according to claim 15, wherein the step of polymerising comprises radical polymerisation.

31. A chiral polymer organocatalyst, obtainable by a process according to claim 15.

32. A process for the production of an asymmetric organic compound, which comprises conducting an asymmetric organic transformation with a chiral polymer organocatalyst according to claim 1.
